(19) United States
(12) Patent Application Publication
(10) Pub. No.: US 2007/0105847 A1
(43) Pub. Date: May 10, 2007

(54) 1,3-Dioxane derivatives and analogues thereof useful in the treatment of i.a. obesity and diabetes

(76) Inventors: Braj Bhushan Lohray, Ahmedabad (IN); Vldya Bhushan Lohray, Gujarat (IN)

Correspondence Address: NIXON & VANDERHYE, PC, 901 NORTH GLEBE ROAD, 11TH FLOOR, ARLINGTON, VA, 22203, US

(21) Appl. No.: 10/585,422

(22) PCT Filed: Jan. 7, 2005
(86) PCT No.: PCT/IN05/00011

§ 371 Date: Aug. 21, 2006

(30) Foreign Application Priority Data
Jan. 9, 2004 (IN) 23/MUM/2004

(51) Int. Cl. A61K 31/5415; A61K 31/538; A61K 31/517; A61K 31/4433; C07D 417/02; C07D 413/02; C07D 407/02

(52) U.S. Cl. 514/224.2; 514/229.8; 514/230.5; 514/266.24; 514/335; 514/452; 544/101; 544/105; 544/52; 544/284; 546/282.4; 549/369

(57) ABSTRACT

The present invention discloses novel compounds of formula (I) and their pharmaceutically useful compositions as modulators of PPAR ligands

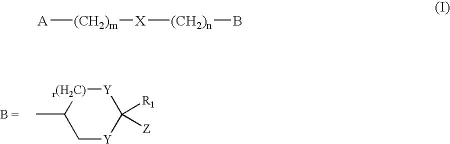

### FIELD OF INVENTION

The present invention relates to novel compounds of the general formula (I), their tautomeric forms, their pharmaceutically acceptable salts, their pharmaceutically acceptable solvates, pharmaceutical compositions containing them, use of these compounds in medicine and the intermediates involved in their preparation.

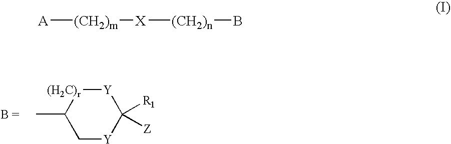

The present invention also relates to a process for the preparation of the compounds of formula (I), their tautomeric forms, their pharmaceutically acceptable salts, their pharmaceutically acceptable solvates, and pharmaceutical compositions containing them.

The compounds of the general formula (I) lower blood glucose, lower or modulate triglyceride levels and/or cholesterol levels and/or low-density lipoproteins (LDL) and raises the high-density lipoproteins (HDL) plasma levels and hence are useful in combating different medical conditions, where such lowering (and raising) is beneficial. Thus, it could be used in the treatment and/or prophylaxis of obesity, hyperlipidaemia, hypercholesteremia, hypertension, atherosclerotic disease events, vascular restenosis, diabetes and many other related conditions.

The compounds of general formula (I) are useful to prevent or reduce the risk of developing atherosclerosis, which leads to diseases and conditions such as artereosclerotic cardiovascular diseases, stroke, coronary heart diseases, cerebrovascular diseases, peripheral vessel diseases and related disorders.

These compounds of general formula (I) are useful for the treatment and/or prophylaxis of metabolic disorders loosely defined as Syndrome X. The characteristic features of Syndrome X include initial insulin resistance followed by hyperinsulinemia, dyslipidemia and impaired glucose tolerance. The glucose intolerance can lead to non-insulin dependent diabetes mellitus (NIDDM, Type 2 diabetes), which is characterized by hyperglycemia, which if not controlled may lead to diabetic complications or metabolic disorders caused by insulin resistance. Diabetes is no longer considered to be associated only with glucose metabolism, but it affects anatomical and physiological parameters, the intensity of which vary depending upon stages/duration and severity of the diabetic state. The compounds of this invention are also useful in prevention, halting or slowing progression or reducing the risk of the above mentioned disorders along with the resulting secondary diseases such as cardiovascular diseases, like arteriosclerosis, atherosclerosis; diabetic retinopathy, diabetic neuropathy and renal disease including diabetic nephropathy, glomerulonephritis, glomerular sclerosis, nephrotic syndrome, hypertensive nephrosclerosis and end stage renal diseases, like microalbuminuria and albuminuria, which may be result of hyperglycemia or hyperinsulinemia.

### BACKGROUND OF THE INVENTION

The present invention discloses compounds suitable for the treatment of hyperlipidemia, diabetes, obesity and similar diseases by modulating the Peroxisome Proliferator Activated Receptor (PPAR). The disease conditions, pathophysiology of the disease conditions, their effects and known & proposed therapies have been described in detail in WO 9119702, WO 9401420, WO 9413650, WO 9503038, WO 9517394, WO 9604260, WO 9604261, WO 9633998, WO 9725042, WO 9736579, WO 9828534, WO 9908501, WO 9916758, WO 9919313, W09920614, WO 0023417, WO 0023445, WO 0023451, WO 0309841, WO 0066572, WO 0116111, WO 0116120, WO 0153257 etc. which are incorporated in their entirety as reference.

Hyperlipidemia has been recognized as the major risk factor in causing cardiovascular diseases due to atherosclerosis [MetS Insights, September; 4, 13-17 (2004)]. Atherosclerosis and other such peripheral vascular diseases affect the quality of life of a large population in the world. The therapy aims to lower the elevated plasma LDL cholesterol, low-density lipoprotein and plasma triglycerides in order to prevent or reduce the risk of occurrence of cardiovascular diseases. The detailed etiology of atherosclerosis and coronary artery diseases is discussed by Ross and Glomset [New Engl. J. Med., 295, 369-377 (1976)].

Peroxisome Proliferator Activated Receptor (PPAR) is a member of the steroid/retinoid/thyroid hormone receptor family. PPAR∝, PPARγ and PPARδ have been identified as subtypes of PPARs. The role of PPAR, in different disease conditions is widely established PPARγ activation has been found to play a central role in initiating and regulating adipocyte differentiation [Endocrinology 135, 798-800, (1994)] and energy homeostasis, [Cell, 83, 803-812 (1995); Cell, 99, 239-242 (1999)]. PPAR□ agonists would stimulate the terminal differentiation of adipocyte precursors and cause morphological and molecular changes characteristic of a more differentiated, less malignant state. During adipocyte differentiation, several highly specialized proteins are induced, which are being involved in lipid storage and metabolism. It is accepted that PPARγ activation leads to expression of CAP gene [Cell Biology, 95, 14751-14756, (1998)], however, the exact link from PPARγ activation to changes in glucose metabolism and decrease in insulin resistance in muscle has not been clear. PPARα is involved in stimulating β-oxidation of fatty acids [Trends Endocrine. Metabolism, 4, 291-296 (1993)] resulting in plasma circulating free fatty acid reduction [Current Biol., 5, 618-621 (1995)]. The role of PPARs in regulation of obesity-related insulin sensitivity and inflammation [Int J Obes Relat Metab Disord. December; 27 Suppl 3:S17-21(2003)], lipid metabolism and insulin sensitivity [Diabetes February;53 Suppl 1:S43-50 (2004)] have been fairly well established. PPARs are also believed to play a role in diseases associated with metabolic syndrome [Curr Top Med Chem., 3(14): 1649-61(2003)]. There is growing evidence that PPAR agonists may also influence the cardiovascular system through PPAR receptors as well as directly by modulating vessel wall function [Diabetes Metab., February; 30(1): 7-12 (2004); Drugs Today (Barc), December;39(12):949-60 (2003)].

PPAR agonists have been found useful in the treatment of obesity [WO 97/36579; Nat Med., April; 10(4):355-61(2004)]. Dual PPAR α and γ agonists have been suggested to be useful for Syndrome X (WO 97/25042). PPAR γ agonists and HMG-CoA reductase inhibitors have exhibited synergism and indicated the usefulness of the combination in the treatment of atherosclerosis and xanthoma [EP 0753 298; Cardiol Rev. May-June; 12(3): 158-70 (2004)].

Leptin is a protein when bound to leptin receptors is involved in sending satiety signal to the hypothalamus. Leptin resistance would therefore lead to excess food in-take, reduced energy expenditure, obesity, impaired glucose tolerance and diabetes [Science, 269, 543-46(1995); Recent Prog Horm Res., 59: 169-205 (2004); Ann NY Acad Sci, June; 967: 363-78 (2002)]. It has been reported that insulin sensitizers lower plasma leptin concentration [Proc. Natl. Acad. Sci. 93, 5793-5796 (1996); WO 98/02159].

Novel heterocyclic compounds which are selective PPAR α agonists have been reported in US 2003/0166697 A1 having the general formula mentioned below which is incorporated herein as reference.
R¹-Het-D-E
wherein:
R¹ is optionally substituted aryl, aromatic heterocyclic group or cycloalkyl group;
Het is an optionally substituted divalent heterocyclic group;
D is alkylene, alkenylene, alkynylene or a group of the formula 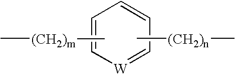
   wherein W is CH or nitrogen;
m is 1-10;
n is 0-9, with the proviso that m+n is 1-10; and
E is a group of the formula 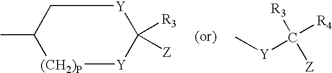
   wherein Y is O or S;
R₃ and R₄ are the same or different and each being H or alkyl;
p is 0-2;
Z is carboxy, alkoxycarbonyl, hydroxymethyl, carbomoyl etc.
   Representative compounds have the following structure:

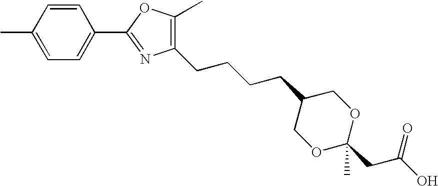

WO 2000004011 discloses compounds having the following general formula for the treatment of dyslipidemia, atherosclerosis and diabetes; 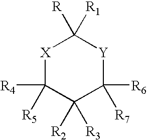
where X, Y═CH₂, O, S, NRa (Ra=H, alkyl, aryl, etc.); R═H, alkyl, cycloalkyl, etc.;
R¹═H, alkyl, hydroxyalkyl, —(CH₂)ₜ-COORc where t=0-6 & Rc represents H or alkyl group, etc.; R₂ & R₃═H, alkyl, cycloalkyl, (C₆-C₁₀)aryl, (C₆-C₁₀)aryl(C₁-C₇)alkyl, 3-10 membered optionally substituted heterocyclic group etc.; or R₂ & R₃ optionally form a chain —(CH2)ᵣ₁(r1=2-5), etc.; R₄-R₇═H, alkyl, (un)substituted aryl, etc.

However, the therapeutic potential of these compounds to treat diseases has not yet been proved and so there remains the need to develop newer medicines which are better or of comparable efficacy with the present treatment regimes, have lesser side effects and require a lower dosage regime

### SUMMARY OF INVENTION

The objective of this invention is to develop novel compounds represented by the general formula (I) used as hypocholesterolemic, hypolipidaemic, hypolipoproteinemic, anti-obesity and antihyperglycemic agents which may have additional body weight lowering effect and beneficial effect in the treatment and/or prophylaxis of diseases caused by hyperlipidaemia, diseases classified under syndrome X and atherosclerosis.

### OBJECTS OF THE INVENTION

The main object of the present invention is to provide novel compounds represented by the general formula (I), their tautomeric forms, their pharmaceutically acceptable salts, their pharmaceutically acceptable solvates, and pharmaceutical compositions containing them or their mixtures thereof.

Yet another object of this invention is to provide a process for the preparation of novel compounds represented by the general formula (I), their tautomeric forms, their pharmaceutically acceptable salts, their pharmaceutically acceptable solvates.

Still another object of the present invention is to provide pharmaceutical compositions containing compounds of the general formula (I), their tautomeric forms, their pharmaceutically acceptable salts, their pharmaceutically acceptable solvates or their mixtures in combination with suitable carriers, solvents, diluents and other media normally employed in preparing such compositions.

### DETAILED DESCRIPTION OF THE INVENTION

Accordingly, the present invention relates to compounds of the general formula (I), 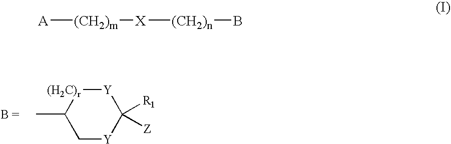
where ‘A’ represents optionally substituted, single or fused aryl, cycloalkyl group or an optionally substituted heteroaryl or an optionally substituted heterocyclyl group;
‘m’=0-2; ‘n’=3-6;
‘X’ represents O, S, —N-(Ra)-0 or —CH₂—;
Ra represents hydrogen, linear or branched, substituted or unsubstituted alkyl, acyl or aryl, aralkyl group;
‘Y’ at each occurrence independently represent O or S; R₁ represents H, linear or branched substituted or unsubstituted alkyl;
r=0-2;
Z represents
—(CH₂)ₛCOOH, alkoxycarbonyl, hydroxymethyl, —CN, substituted or unsubstituted tetrazoles, alkylcarbonyl groups, s=0-4;

When ‘A’ is substituted, suitable substitutions on ‘A’ may be selected from hydroxyl, oxo, halo, thio, nitro, amino, cyano, formyl, or substituted or unsubstituted groups selected from amidino, alkyl, haloalkyl, perhaloalkyl, alkoxy, haloalkoxy, perhaloalkoxy, alkenyl, alkynyl, cycloalkyl, cycloalkenyl, bicycloalkyl, bicycloalkenyl, alkoxy, alkenoxy, cycloalkoxy, aryl, aryloxy, aralkyl, aralkoxy, acyl, acyloxy, acylamino, monosubstituted or disubstituted amino, arylamino, aralkylamino, carboxylic acid and its derivatives such as esters and amides, carbonylamino, hydroxyalkyl, aminoalkyl, alkoxyalkyl, aryloxyalkyl, aralkoxyalkyl, alkylthio, thioalkyl, arylthio, alkylsulfonylamino, alkylsulfonyloxy, alkoxycarbonylamino, aryloxycarbonylamino, aralkyloxycarbonylamino, aminocarbonylamino, alkylaminocarbonylamino, alkoxyamino, hydroxyl amino, sulfenyl derivatives, sulfonyl derivatives,

with the proviso that when X═CH₂ and

i) ‘A’ represents substituted heterocyclic group, the substitutions on ‘A’ does not represent aryl, aromatic, heterocyclic or cycloalkyl group; and
ii) ‘A’ represents substituted aryl group, the substituent on ‘A’ represents alkylsulfonyloxy, aryloxy, aralkoxy, cycloalkyl, heteroaryl or heterocyclic group.

Suitable substitutions on ‘B’ may be selected from hydroxyl, oxo, halo, thio, nitro, amino, cyano, formyl, or substituted or unsubstituted groups selected from alkyl, haloalkyl, aryl groups.

Suitable substitutions on any of the substituents on ‘A’ & ‘B’ may be selected from hydroxyl, oxo, halo, thio, nitro, amino, cyano, formyl, or substituted or unsubstituted groups selected from amidino, alkyl, haloalkyl, perhaloalkyl, alkoxy, haloalkoxy, perhaloalkoxy, alkenyl, alkynyl, cycloalkyl, cycloalkenyl, bicycloalkyl, bicycloalkenyl, alkoxy, alkenoxy, cycloalkoxy, acyl, acyloxy, acylamino, monosubstituted or disubstituted amino, arylamino, aralkylamino, carboxylic acid and its derivatives such as esters and amides, carbonylamino, hydroxyalkyl, aminoalkyl, alkoxycarbonylamino, aryloxycarbonylamino, aralkyloxycarbonylamino, aminocarbonylamino, alkylamiinocarbonylamino, alkoxyamino, hydroxyl amino groups.

The term “substituted” used alone or in combination with other radicals, denotes suitable substituents on that radical such as substituted alkyl, substituted alkenyl, substituted alkynyl, substituted cycloalkyl, substituted aryl, etc, mentioned anywhere in the specification. The suitable substituents include, but are not limited to the following radicals, alone or in combination with other radicals, such as, hydroxyl, oxo, halo, thio, nitro, amino, cyano, formyl, amidino, alkyl, haloalkyl, perhaloalkyl, alkoxy, haloalkoxy, perhaloalkoxy, alkenyl, alkynyl, cycloalkyl, cycloalkenyl, bicycloalkyl, bicycloalkenyl, alkoxy, alkenoxy, cycloalkoxy, acyl, acyloxy, acylamino, monosubstituted or disubstituted amino, arylamino, aralkylamino, carboxylic acid and its derivatives such as esters and amides, carbonylamino, hydroxyalkyl, aminoalkyl, alkoxyalkyl, alkoxycarbonylamino, aryloxycarbonylamino, aralkyloxycarbonylamino, sulfonic acid and its derivatives.

The various groups, radicals and substituents used anywhere in the specification are described in the following paragraphs.

The term “alkyl” used herein, either alone or in combination with other radicals, denotes a linear or branched radical containing one to twelve carbons, such as methyl, ethyl, n-propyl, iso-propyl, n-butyl, sec-butyl, tert-butyl, amyl, t-amyl, n-pentyl, n-hexyl, iso-hexyl, heptyl, octyl and the like.

The term “alkenyl” used herein, either alone or in combination with other radicals, denotes a linear or branched radical containing two to twelve carbons such as vinyl, allyl, 2-butenyl, 3-butenyl, 2-pentenyl, 3-pentenyl, 4-pentenyl, 2-hexenyl, 3-hexenyl, 4-hexenyl, 5-hexenyl, 2-heptenyl, 3-heptenyl, 4-heptenyl, 5-heptenyl, 6-heptenyl and the like. The term “alkenyl” includes dienes and trienes of straight and branched chains.

The term “alkynyl” used herein, either alone or in combination with other radicals, denotes a linear or branched radical containing two to twelve carbons, such as ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 3-hexynyl, 4-hexynyl, 5-hexynyl, and the like. The term “alkynyl” includes di- and tri-ynes.

The term “cycloalkyl” used herein, either alone or in combination with other radicals, denotes a radical containing three to seven carbons, such as cyclopropyl, cyclobutyl, cyclopentyl, cyclohexyl, cycloheptyl and the like.

The term “cycloalkenyl” used herein, either alone or in combination with other radicals, denotes a radical containing three to seven carbons, such as cyclopropenyl, 1-cyclobutenyl, 2-cylobutenyl, 1-cyclopentenyl, 2-cyclopentenyl, 3-cyclopentenyl, 1-cyclohexenyl, 2-cyclohexenyl, 3-cyclohexenyl, 1-cycloheptenyl, cycloheptadienyl, cycloheptatrienyl, and the like.

The term “alkoxy” used herein, either alone or in combination with other radicals, denotes an alkyl radical, as defined above, attached directly to an oxygen atom, such as methoxy, ethoxy, n-propoxy, iso-propoxy, n-butoxy, t-butoxy, iso-butoxy, pentyloxy, hexyloxy, and the like.

The term “alkenoxy” used herein, either alone or in combination with other radicals, denotes an alkenyl radical, as defined above, attached to an oxygen atom, such as vinyloxy, allyloxy, butenoxy, pentenoxy, hexenoxy, and the like.

The term “cycloalkoxy” used herein, either alone or in combination with other radicals, denotes a cycloalkyl radical as defined above, attached directly to an oxygen atom, such as cyclopropyloxy, cyclobutyloxy, cyclopentyloxy, cyclohexyloxy, cycloheptyloxy and the like.

The term “halo” or “halogen” used herein, either alone or in combination with other radicals, such as “haloalkyl”, “perhaloalkyl” etc refers to a fluoro, chloro, bromo or iodo group. The term “haloalkyl” denotes a alkyl radical, as defined above, substituted with one or more halogens; such as perhaloalkyl, more preferably, perfluoro(C₁-C₆)alkyl such as fluoromethyl, difluoromethyl, trialuoromethyl, fluoroethyl, difluoroethyl, trifluoroethyl, mono or polyhalo substituted methyl, ethyl, propyl, butyl, pentyl or hexyl groups. The term “haloalkoxy” denotes a haloalkyl, as defined above, directly attached to an oxygen atom, such as fluoromethoxy, chloromethoxy, fluoroethoxy chloroethoxy groups, and the like. The term “perhaloalkoxy” denotes a perhaloalkyl radical, as defined above, directly attached to an oxygen atom, trifluoromethoxy, trifluoroethoxy, and the like.

The term “aryl” or “aromatic” used herein, either alone or in combination with other radicals, denotes an aromatic system containing one, two or three rings wherein such rings may be attached together in a pendant manner or may be fused, such as phenyl, naphthyl, tetrahydronaphthyl, indane, biphenyl, and the like. The term ‘aralkyl” denotes an alkyl group, as defined above, attached to an aryl, such as benzyl, phenethyl, naphthylmethyl, and the like. The term “aryloxy” denotes an aryl radical, as defined above, attached to an alkoxy group, such as phenoxy, naphthyloxy and the like, which may be substituted. The term “aralkoxy” denotes an arylalkyl moiety, as defined above, such as benzyloxy, phenethyloxy, naphthylmethyloxy, phenylpropyloxy, and the like, which may be substituted.

The term “heterocyclyl” or “heterocyclic” used herein, either alone or in combination with other radicals, denotes saturated, partially saturated and unsaturated ring-shaped radicals, the heteroatoms selected from nitrogen, sulfur and oxygen. Examples of saturated heterocyclic radicals include aziridinyl, azetidinyl, pyrrolidinyl, imidazolidinyl, piperidinyl, piperazinyl, 2-oxopiperidinyl, 4-oxopiperidinyl, 2-oxopiperazinyl, 3-oxopiperazinyl, morpholinyl, thiomorpholinyl, 2-oxomorpholinyl, azepinyl, diazepinyl, oxapinyl, thiazepinyl, oxazolidinyl, thiazolidinyl, and the like; examples of partially saturated heterocyclic radicals include dihydrothiophene, dihydropyran, dihydrofuran, dihydrothiazole, and the like.

The term “heteroaryl” or “heteroaromatic” used herein, either alone or in combination with other radicals, denotes unsaturated 5 to 6 membered heterocyclic radicals containing one or more hetero atoms selected from O, N or S, such as pyridyl, thienyl, furyl, pyrrolyl, oxazolyl, thiazolyl, isothiazolyl, imidazolyl, isoxazolyl, oxadiazolyl, thiadiazolyl, triazolyl, tetrazolyl, benzopyranyl, benzopyranonyl, benzofuranyl, benzothienyl, indolinyl, indolyl, azaindolyl, azaindolinyl, benzodihydrofuranyl, benzodihydrothienyl, pyrazolopyrimidinyl, pyrazolopyrimidonyl, azaquinazolinyl, azaquinazolinoyl, pyridofuranyl, pyridothienyl, thienopyrimidyl, thienopyrimidonyl, quinolinyl, pyrimidinyl, pyrazolyl, quinazolinyl, quinazolonyl, pyrimidonyl, pyridazinyl, triazinyl, benzoxazinyl, benzoxazinonyl, benzothiazinyl, benzothiazinonyl, benzoxazolyl, benzothiazolyl, benzimidazolyl, benzotriazolyl, phthalazynil, naphthylidinyl, purinyl, carbazolyl, phenothiazinyl, phenoxazinyl, and the like.

The term “acyl” used herein, either alone or in combination with other radicals, denotes a radical containing one to eight carbons such as formyl, acetyl, propanoyl, butanoyl, iso-butanoyl, pentanoyl, hexanoyl, heptanoyl, benzoyl and the like, which may be substituted.

The term “acyloxy” used herein, either alone or in combination with other radicals, denotes a radical acyl, as defined above, directly attached to an oxygen atom, such as acetyloxy, propionyloxy, butanoyloxy, iso-butanoyloxy, benzoyloxy and the like.

The term “acylamino” used herein, either alone or in combination with other radicals, denotes an acyl group as defined earlier, may be CH₃CONH, C₂H₅CONH, C₃H7CONH C₄H₉CONH, C₆H₅CONH and the like, which may be substituted.

The term “mono-substituted amino” used herein, either alone or in combination with other radicals, denotes an amino group, substituted with one group selected from (C₁-C₆)alkyl, substituted alkyl, aryl, substituted aryl or arylalkyl groups. Examples of monoalkylamino group include methylamine, ethylamine, n-propylamine, n-butylamine, n-pentylamine and the like.

The term ‘disubstituted amino” used herein, either alone or in combination with other radicals, denotes an amino group, substituted with two radicals that may be same or different selected from (C₁C₆)alkyl, substituted alkyl, aryl, substituted aryl, or arylalkyl groups, such as dimethylamino, methylethylamino, diethylamino, phenylmethyl amino and the like.

The term “arylamino” used herein, either alone or in combination with other radicals, denotes an aryl group, as defined above, linked through amino having a free valence bond from the nitrogen atom, such as phenylamino, naphthylamino, N-methyl anilino and the like.

The term “aralkylamino” used herein, either alone or in combination with other radicals, denotes an arylalkyl group as defined above linked through amino having a free valence bond from the nitrogen atom e.g. benzylamino, phenethylamino, 3-phenylpropylamino, 1-napthylmethylamino, 2-(1-napthyl)ethylamino and the like.

The term “oxo” or “carbonyl” used herein, either alone (—C═O—) or in combination with other radicals, such as “alkylcarbonyl”, denotes a carbonyl radical (13 C═O—) substituted with an alkyl radical such as acyl or alkanoyl, as described above.

The term “carboxylic acid” used herein, alone or in combination with other radicals, denotes a —COOH group, and includes derivatives of carboxylic acid such as esters and amides. The term “ester” used herein, alone or in combination with other radicals, denotes —COO— group, and includes carboxylic acid derivatives, where the ester moieties are alkoxycarbonyl, such as methoxycarbonyl, ethoxycarbonyl, and the like, which may be substituted; aryloxycarbonyl group such as phenoxycarbonyl, napthyloxycarbonyl, and the like, which may be substituted; aralkoxycarbonyl group such as benzyloxycarbonyl, phenethyloxycarbonyl, napthylmethoxycarbonyl, and the like, which may be substituted;

The term “amide” used herein, alone or in combination with other radicals, represents an aminocarbonyl radical (H₂N—C═O—), wherein the amino group is mono- or di-substituted or unsubstituted, such as methylamide, dimethylamide, ethylamide, diethylamide, and the like. The term “aminocarbonyl” used herein, either alone or in combination with other radicals, with other terms such as ‘aminocarbonylalkyl”, “n-alkylaminocarbonyl”, “N-arylaminocarbonyl”, “N,N-dialkylaminocarbonyl”, “N-alkyl-N-arylaminocarbonyl”, “N-alkyl-N-hydroxyaminocarbonyl”, and “N-alkyl-N-hydroxyaminocarbonylalkyl”, substituted or unsubstituted. The terms “N-alkylaminocabonyl” and “N,N-dialkylaminocarbonyl” denotes aminocarbonyl radicals, as defined above, which have been substituted with one alkyl radical and with two alkyl radicals, respectively. Preferred are “lower alkylaminocarbonyl” having lower alkyl radicals as described above attached to aminocarbonyl radical. The terms “N-arylaminocarbonyl” and “N-alkyl-N-arylaminocarbonyl” denote amiocarbonyl radicals substituted, respectively, with one aryl radical, or one alkyl, and one aryl radical. The term “aminocarbonylalkyl” includes alkyl radicals substituted with aminocarbonyl radicals.

The term “hydroxyalkyl” used herein, either alone or in combination with other radicals, denotes an alkyl group, as defined above, substituted with one or more hydroxy radicals, such as hydroxymethyl, hydroxyethyl, hydroxypropyl, hydroxybutyl, hydroxypentyl, hydroxyhexyl and the like.

The term “aminoalkyl” used herein, alone or in combination with other radicals, denotes an amino (—NH₂) moiety attached to an alkyl radical, as defined above, which may be substituted, such as mono- and di-substituted aminoalkyl. The term “alkylamino” used herein, alone or in combination with other radicals, denotes an alkyl radical, as defined above, attached to an amino group, which may be substituted, such as mono- and di-substituted alkylamino.

The term “alkoxyalkyl” used herein, alone or in combination with other radicals, denotes an alkoxy group, as defined above, attached to an alkyl group, such as methoxymethyl, ethoxymethyl, methoxyethyl, ethoxyethyl and the like. The term “aryloxyalkyl” used herein, alone or in combination with other radicals, includes phenoxymethyl, napthyloxymethyl, and the like. The term “aralkoxyalkyl” used herein, alone or in combination with other radicals, includes C₆H₅CH₂OCH₂, C₆H₅CH₂OCH₂CH₂, and the like.

The term “alkylthio” used herein, either alone or in combination with other radicals, denotes a straight or branched or cyclic monovalent substituent comprising an alkyl group of one to twelve carbon atoms, as defined above, linked through a divalent sulfur atom having a free valence bond from the sulfur atom, such as methylthio, ethylthio, propylthio, butylthio, pentylthio and the like. Examples of cyclic alkylthio are cyclopropylthio, cyclobutylthio, cyclopentylthio, cyclohexylthio and the like, which may be substituted.

The term “thioalkyl” used herein, either alone or in combination with other radicals, denotes an alkyl group, as defined above, attached to a group of formula —SR′, where R′ represents hydrogen, alkyl or aryl group, e.g. thiomethyl, methylthiomethyl, phenylthiomethyl and the like, which may be substituted.

The term “arylthio’ used herein, either alone or in combination with other radicals, denotes an aryl group, as defined above, linked through a divalent sulfur atom, having a free valence bond from the sulfur atom such as phenylthio, napthylthio and the like.

The term “alkoxycarbonylamino” used herein, alone or in combination with other radicals, denotes an alkoxycarbonyl group, as defined above, attached to an amino group, such as methoxycarbonylamino, ethoxycarbonylamino, and the like. The term “aryloxycarbonylamino” used herein, alone or in combination with other radicals, denotes an aryloxycarbonyl group, as defined above, attached to the an amino group, such as C₆H₅OCONH, C₆H₅OCONCH₃, C₆H₅OCONC₂H₅, C₆H₄(CH₃O )CONH, C₆H4(OCH₃)OCONH, and the like. The term “aralkoxycarbonylamino” used herein, alone or in combination with other radicals, denotes an aralkoxycarbonyl group, as defined above, attached to an amino group C₆H₅CH₂OCONH, C₆H₅CH₂CH₂CH₂OCONH, C₆H₅CH₂OCONHCH₃, C₆H₅CH₂OCONC₂H₅, C₆H₄(CH₃)CH₂OCONH, C₆H₄(OCH₃)CH₂OCONH and the like.

The term “aminocarbonylamino”, “alkylaminocarbonylamino”, “dialkylaminocarbonylamino” used herein, alone or in combination with other radicals, denotes a carbonylamino (—CONH₂) group, attached to amino(NH2), alkylamino group or dialkylamino group respectively, where alkyl group is as defined above.

The term “amidino” used herein, either alone or in combination with other radicals, denotes a —C(═NH)—NH₂ radical. The term “alkylamidino” denotes an alkyl radical, as discussed above, attached to an amidino group.

The term “alkoxyamino” used herein, alone or in combination with other radicals, denotes an alkoxy group, as defined above, attached to an amino group. The term “hydroxyamino” used herein, alone or in combination with other radicals, denotes —NHOH moiety, and may be substituted.

The term “sulfenyl” or “sulfenyl and its derivatives” used herein, alone or in combination with other radicals, denotes a bivalent group, —SO— or RₓSO, where Rₓ is substituted or unsubstituted alkyl, aryl, heteroaryl, heterocyclyl, and the like.

The term “sulfonyl” or “sulfones and its derivatives” used herein, either alone or in combination with other radicals, with other terms such as alkylsulfonyl, denotes divalent radical —SO₂—, or RₓSO₂—, where Rₓ is substituted or unsubstituted groups selected from alkyl, aryl, heteroaryl, heterocyclyl, and the like. “Alkylsulfonyl” denotes alkyl radicals, as defined above, attached to a sulfonyl radical, such as methylsulfonyl, ethylsulfonyl, propylsulfonyl and the like. The term “arylsulfonyl” used herein, either alone or in combination with other radicals, denotes aryl radicals, as defined above, attached to a sulfonyl radical, such as phenylsulfonyl and the like.

Suitable groups and substituents on the groups may be selected from those described anywhere in the specification.

Particularly useful compounds may be selected from
Methyl-5-[4-(2-ethyl-4-oxo-4H-quinazolin-3-yl)-butyl]-2-methyl-[1,3]dioxane-2-carboxylate;
Methyl-5-[4-(2-ethyl-quinazolin-4-yloxy)-butyl]-2-methyl-[1,3]dioxane-2-carboxylate;
Methyl-5-[6-(4-chloro-phenyl)-5-(4-methylsulfanyl-phenyl)-6-oxo-hexyl]-2-methyl-[1,3]dioxane-2-carboxylate;
Methyl-5-[4-(2,3-dihydro-benzo[1,4]oxazin-4-yl)-butyl]-2-methyl-[1,3]dioxane-2-carboxylate;
Methyl-2-methyl-5-(4-phenoxazin-10-yl-butyl)-[1,3]dioxane-2-carboxylate;
Methyl-5-[4-(6,7-dihydro-4H-thieno[3,2-c]pyridin-5-yl)-butyl]-2-methyl-[1,3]dioxane-2-carboxylate;
Methyl-5-(4-carbazol-9-yl-butyl)-2-methyl-[1,3]dioxane-2-carboxylate;
Methyl-2-methyl-5-[4-(3-oxo-2,3-dihydro-benzo[1,4]thiazin-4-yl)-butyl]-[1,3]dioxane-2-carboxylate;
Methyl-5-[4-(2,3-dihydro-benzo[1,4]thiazin-4-yl)-butyl]-2-methyl-[1,3]dioxane-2-carboxylate;
Methyl-2-methyl-5-(4-phenothiazin-10-yl-butyl)-[1,3]dioxane-2-carboxylate;
Methyl-5-(4-indol-1-yl-butyl)-2-methyl-[1,3]dioxane-2-carboxylate;
Methyl-2-methyl-5-(5-phenyl-5-pyridin-4-yl-pentyl)-[1,3]dioxane-2-carboxylate;
Methyl-5-[4-(4-benzyl-phenoxy)-butyl]-2-methyl-[1,3]dioxane-2-carboxylate;
Methyl-2-methyl-5-[4-(3-oxo-2,3-dihydro-benzo[1,4]oxazin-4-yl)-butyl]-[1,3]dioxane-2-carboxylate;
Methyl-5-{(4-[2-(2-hydroxy-ethyl)-3-oxo-2,3-dihydro-benzo[1,4]oxazin-4-yl]-butyl}-2-methyl-[1,3]dioxane-2-carboxylate;
Methyl-2-methyl-5-[4-(4-phenoxy-phenoxy)-butyl]-[1,3]dioxane-2-carboxylate;
Methyl-5-(3-benzo[1,3]dioxol-5-yl-propyl)-2-methyl-[1,3]dioxane-2-carboxylate;
Methyl-5-[4-(4-methanesulfonyloxy-phenyl)-butyl]-2-methyl-[1,3]dioxane-2-carboxylate;
Methyl-5-[4-(4-benzyloxy-phenyl)-butyl]-2-methyl-[1,3]dioxane-2-carboxylate;
Methyl-2-methyl -5-(3-phenylsulfanyl-propyl)-[1,3]dioxane-2-carboxylate;
Ethyl-5-[3-(4-bromo-phenoxy)-propyl]-2-methyl-[1,3]dioxane-2-carboxylate;
Methyl-2-methyl-5-[3-(4-phenoxy-phenoxy)-propyl]-[1,3 ]dioxane-2-carboxylate;
Methyl-5-[3-(4-isopropyl-phenoxy)-propyl]-2-methyl-[1,3 ]dioxane-2-carboxylate;
Methyl-2-methyl-5-(3-p-tolyloxy-propyl)-[1,3]dioxane-2-carboxylate;
Methyl-5-[3-(4-bromo-phenylsulfanyl)-propyl]-2-methyl-[1,3]dioxane-2-carboxylate;
Methyl-2-methyl-5-(3-phenoxy-propyl)-[1,3]dioxane-2-carboxylate;
Methyl-5-[3-(4-fluoro-phenoxy)-propyl]-2-methyl-[1,3]dioxane-2-carboxylate;
Methyl-2-methyl-5-[3-(naphthalen-2-yloxy)-propyl]-[1,3]dioxane-2-carboxylate;
Methyl-5-[3-(4-benzyloxy-phenoxy)-propyl]-2-methyl-[1,3]dioxane-2-carboxylate;
Methyl-5-[3-(4-methoxy-phenoxy)-propyl]-2-methyl-[1,3]dioxane-2-carboxylate;
Methyl-5-[3-(4-benzyl-phenoxy)-propyl]-2-methyl-[1,3]dioxane-2-carboxylate;
5-[4-(2-Ethyl-4-oxo-4H-quinazolin-3-yl)-butyl]-2-methyl-[1,3]dioxane-2-carboxylic acid and its pharmaceutically acceptable salts;
5-[4-(2-Ethyl-quinazolin-4-yloxy)-butyl]-2-methyl-[1,3]dioxane-2-carboxylic acid and its pharmaceutically acceptable salts;
5-[6-(4-Chloro-phenyl)-5-(4-methylsulfanyl-phenyl)-6-oxo-hexyl]-2-methyl-[1,3]dioxane-2-carboxylic acid and its pharmaceutically acceptable salts;
5-[4-(2,3-Dihydro-benzo[1,4]oxazin-4-yl)-butyl]-2-methyl-[1,3]dioxane-2-carboxylic acid and its pharmaceutically acceptable salts;
2-Methyl-5-(4-phenoxazin-10-yl-butyl)-[1,3]dioxane-2-carboxylic acid and its pharmaceutically acceptable salts;
5-(4-Carbazol-9-yl-butyl)-2-methyl-[1,3]dioxane-2-carboxylic acid and its pharmaceutically acceptable salts;
2-Methyl-5-[4-(3-oxo-2,3-dihydro-benzo[1,4]thiazin-4-yl)-butyl]-[1,3]dioxane-2-carboxylic acid and its pharmaceutically acceptable salts;
5-[4-(2,3-Dihydro-benzo [1,4]thiazin-4-yl)-butyl]-2-methyl-[1,3]dioxane-2-carboxylic acid and its pharmaceutically acceptable salts;
2-Methyl-5-(4-phenothiazin-10-yl-butyl)-[1,3]dioxane-2-carboxylic acid and its pharmaceutically acceptable salts;
5-(4-Indol-1-yl-butyl)-2-methyl-[1,3]dioxane-2-carboxylic acid and its pharmaceutically acceptable salts;
2-Methyl-5-(5-phenyl-5-pyridin-4-yl-pentyl)-[1,3]dioxane-2-carboxylic acid and its pharmaceutically acceptable salts;
5-[4-(4-Benzyl-phenoxy)-butyl]-2-methyl-[1,3]dioxane-2-carboxylic acid and its pharmaceutically acceptable salts;
2-Methyl-5-[4-(3-oxo-2,3-dihydro-benzo[1,4]oxazin-4-yl)-butyl]-[1,3]dioxane-2-carboxylic acid and its pharmaceutically acceptable salts;
5-{4-[2-(2-Hydroxy-ethyl)-3-oxo-2,3-dihydro-benzo[1,4]oxazin-4-yl]-butyl}-2-methyl-[1,3]dioxane-2-carboxylic acid and its pharmaceutically acceptable salts;
2-Methyl-5-[4-(4-phenoxy-phenoxy)-butyl]-[1,3]dioxane-2-carboxylic acid and its pharmaceutically acceptable salts;
5-(3-Benzo[1,3]dioxol-5-yl-propyl)-2-methyl-[1,3]dioxane-2-carboxylic acid and its pharmaceutically acceptable salts;
5-[4-(4-Methanesulfonyloxy-phenyl)-butyl]-2-methyl-[1,3]dioxane-2-carboxylic acid and its pharmaceutically acceptable salts;
5-[4-(4-Benzyloxy-phenyl)-butyl]-2-methyl-[1,3]dioxane-2-carboxylic acid and its pharmaceutically acceptable salts;
2-Methyl-5-(3-phenylsulfanyl-propyl)-[1,3]dioxane-2-carboxylic acid and its pharmaceutically acceptable salts;
5-[3-(4-Bromo-phenoxy)-propyl]-2-methyl-[1,3]dioxane-2-carboxylic acid and its pharmaceutically acceptable salts;
2-Methyl-5-[3-(4-phenoxy-phenoxy)-propyl]-[1,3]dioxane-2-carboxylic acid and its pharmaceutically acceptable salts;
5-[3-(4-Isopropyl-phenoxy)-propyl]-2-methyl-[1,3]dioxane-2-carboxylic acid and its pharmaceutically acceptable salts;
2-Methyl-5-(3-p-tolyloxy-propyl)-[1,3]dioxane-2-carboxylic acid and its pharmaceutically acceptable salts;
5-[3-(4-Bromo-phenylsulfanyl)-propyl]-2-methyl-[1,3]dioxane-2-carboxylic acid and its pharmaceutically acceptable salts;
2-Methyl-5-(3-phenoxy-propyl)-[1,3]dioxane-2-carboxylic acid and its pharmaceutically acceptable salts;
5-[3-(4-Fluoro-phenoxy)-propyl]-2-methyl-[1,3]dioxane-2-carboxylic acid and its pharmaceutically acceptable salts;
2-Methyl-5-[3-(naphthalen-2-yloxy)-propyl]-[1,3]dioxane-2-carboxylic acid and its pharmaceutically acceptable salts;
5-[3-(4-Benzyloxy-phenoxy)-propyl]-2-methyl-[1,3]dioxane-2-carboxylic acid and its pharmaceutically acceptable salts;
5-[3-(4-Methoxy-phenoxy)-propyl]-2-methyl-[1,3]dioxane-2-carboxylic acid and its pharmaceutically acceptable salts;
5-[3-(4-Benzyl-phenoxy)-propyl]-2-methyl-[1,3]dioxane-2-carboxylic acid and its pharmaceutically acceptable salts;

The novel compounds of this invention may be prepared using the reactions and techniques described in this section. The reactions are performed in solvents appropriate to the reagents and materials employed and are suitable for the transformations being effected. It is understood by those skilled in the art that the nature and order of the synthetic steps presented may be varied for the purpose of optimizing the formation of the compounds of the present invention.

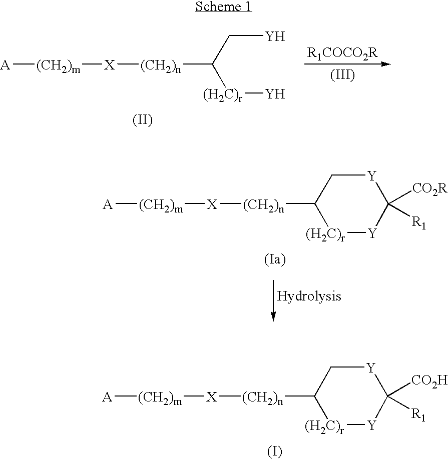

The compounds of general formula (I) wherein all the symbols are as defined earlier, may be prepared by route outlined in scheme 1 above which comprises
i) Reacting a compound of formula (II) with a compound of formula (III) to obtain compound of formula (Ia) wherein R represents alkyl group and all other symbols are as defined earlier. Generally, the reaction may be carried out in an appropriate solvent selected from polar solvents such as acetonitrile, DMP and the like, ether solvents such as THF, dioxane, diethyl ether, dimethoxy ethane and the like, halogenated solvents like CHCl₃ or dichloromethane, dichloroethane and the like, hydrocarbon solvents such as benzene, toluene, n-hexane, cyclohexane and the like, ester solvents such as methyl acetate, ethyl acetate, isopropyl acetate and the like or mixtures thereof, in the presence of Lewis acid such as boron trifluoride diethyl ether complex at −22 to 120° C. The reaction may be carried out in the atmosphere of an inert gas such as nitrogen. The reaction time may vary from 30 minutes to 24 hours.
ii) Hydrolyzing a compound of formula (Ia) with suitable reagents/solvents to a compound of formula (I) wherein all the symbols are as defined earlier. Suitable hydrolyzing solvents may be selected from alcoholic solvents like methanol, ethanol, propanol, isopropanol, t-butanol and the like or mixtures thereof, and water in the presence of suitable acids or bases at −20 to 100° C. Suitable acids may be HCl, PTSA and the like; suitable bases may be LiOH, NaOH, KOH and the like.
iii) optionally, if desired, the compounds of formula (I) are converted to their suitable pharmaceutically acceptable salts by techniques known in the art.

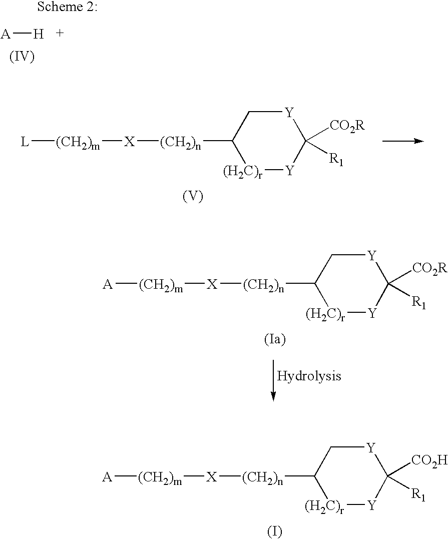

Alternatively, the compounds of general formula (I) wherein all the symbols are as defined earlier may be prepared by route outlined in scheme 2 above which comprises;
i) Reacting the compound of general formula (V) where L represents a suitable leaving group such as halogen, mesylate, tosylate, triflate & the like, with compounds of general formula (IV) to obtain the compound of general formula (Ia). Suitable bases like metal hydrides e.g NaH and the like, alkali metal carbonates e.g. potassium carbonate, sodium carbonate and the like, sodium hydroxide, potassium hydroxide, organic bases e.g. trialkyl amines and the like, organolithium reagents e.g. butyllithium, lithium diisopropylamide, lithium hexamethyldisilazide and the like may be used. Reaction may be carried out in suitable solvents like DMF, DMSO, THF, dioxane, n-hexane, cyclohexane, dichloroethane, acetone, dichloromethane, toluene and the like or mixture thereof based on the suitability for the bases used. Reaction temperature may range from −78° C. to the reflux temperature of the solvent(s) used. Inert atmosphere may optionally be maintained using N₂, He, or argon gas. Reaction time may range from 1 to 72 hours.
ii) Hydrolyzing a compound of formula (Ia) with suitable reagents/solvents to a compound of formula (I) wherein all the symbols are as defined earlier. Suitable hydrolyzing solvents may be selected from alcoholic solvents like methanol, ethanol, propanol, isopropanol, t-butanol and the like or mixtures thereof, and water in the presence of suitable acids or bases at −20 to 100° C. Suitable acids may be HCl, PTSA and the like; suitable bases may be NaOH, KOH and the like.
iii) optionally, if desired, the compounds of formula (I) are converted to their suitable pharmaceutically acceptable salts by techniques known in the art.

It will be appreciated that in any of the above mentioned reactions any reactive group in the substrate molecule may be protected, according to conventional chemical practice. Suitable protecting groups in any of the above mentioned reactions are those used conventionally in the art. The methods of formation and removal in such protecting groups are those conventional methods appropriate to the molecule being protected. T. W. Greene and P. G. M. Wuts “Protective groups in Organic Synthesis”, John Wiley & Sons, Inc, 1999, 3^{rd} Ed., along with references therein.

It will be appreciated that when substituents have different sites where they can be attached, such differently attached substituents are also included in the present invention.

The novel compounds of the present invention can be formulated into suitable pharmaceutically acceptable compositions by combining with suitable excipients by techniques and processes and concentrations as are well known.

The compounds of formula (I) or pharmaceutical compositions containing them may be administered either by oral, topical or parenteral administration.

The pharmaceutical composition is provided by employing conventional techniques. Preferably the composition is in unit dosage form containing an effective amount of the active component, that is, the compounds of formula (I) according to this invention.

The quantity of active component, that is, the compounds of formula (I) according to this invention, in the pharmaceutical composition and unit dosage form thereof may be varied or adjusted widely depending upon the particular application method, the potency of the particular compound and the desired concentration. Generally, the quantity of active component will range between 0.5% to 90% by weight of the composition.

The compounds of general formula (I) or the compositions thereof are useful for the treatment and/or prophylaxis of disease caused by metabolic disorders such as hyperlipidemia, insulin resistance, leptin resistance, Syndrome X hyperglycemia, obesity, or inflammation.

The invention is explained in greater detail by the examples given below, which are provided by way of illustration only and therefore should not be construed to limit the scope of the invention.

It will be appreciated that one or more of the processes described in the general schemes above may be used to prepare the compounds of the present invention. 1H NMR spectral data given in the tables (vide infra) are recorded using a 300 MHz spectrometer (Bruker AVANCE-300) and reported in δ scale. Until and otherwise mentioned the solvent used for NMR is CDCl₃ using Tetramethyl silane as the internal standard.

### Preparation 1

### Methyl-2-methyl-5-(5-phenyl-5-pyridin-4-yl-pentyl)-[1,3]dioxane-2-carboxylate. (compound No. 12)

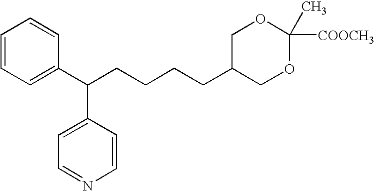

A solution of 4-benzyl pyridine (250 mg) in dry tetrahydrofuran (3 mL) was cooled to −78° C. and 2.35 mL of 1M solution of lithium hexamethyldisilazide in tetrahydrofuran was added. After stirring for one hour at the same temperature another solution of Methyl-5-(4-iodo-butyl)-2-methyl-[1,3]dioxane-2-carboxylate (500 mg) in tetrahydrofuran (3 mL) was added and the reaction mixture was stirred for 3 hours allowing the temperature to rise to 30° C. The reaction mixture was poured in to ice cold water (25 mL) and extracted with ethyl acetate (3×10 mL). The combined organic extract was washed with water (25 mL), brine solution (25 mL), dried over sodium sulfate and evaporated under reduced pressure. The crude product obtained was flash chromatographed over silicagel using 10% ethyl acetate in petroleum ether as eluent to yield 233 mg of the product.

### Preparation 2

### Methyl-5-(4-indol-1-yl-butyl)-2-methyl-[1,3]dioxane-2-carboxylate (compound No. 11)

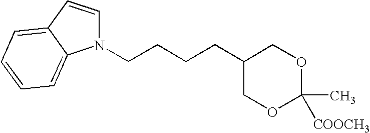

A solution of indole (340 mg) in dimethyl sulfoxide (3 mL) was added to an ice cold suspension of potassium hydroxide (326 mg) in dimethyl sulfoxide (3 mL). After stirring for 10 minutes a solution of methyl-5-(4-iodo-butyl)-2-methyl-[1,3]dioxane-2-carboxylate (1.0 g) in dimethyl sulfoxide (5 mL) was added and the reaction mixture was stirred for 5 hours at 30° C. Reaction mixture was poured in to ice cold water (50 mL) and extracted with ethyl acetate (3×20 mL). The combined organic extract was washed with water (50 mL), brine. solution (50 mL), dried over sodium sulfate and evaporated under reduced pressure. Crude product was flash chromatographed over silica gel using 10% ethyl acetate in petroleum ether as eluent to yield 400 mg of product.

### Preparation 3

### Methyl-2-methyl-5-[4-(3-oxo-2,3-dihydro-benzo[1,4]oxazin-4-yl)-butyl]-[1,3]dioxane-2-carboxylate (compound No. 14)

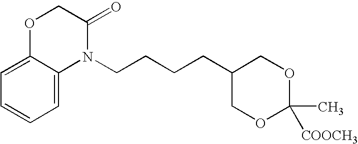

To a stirred suspension of cesium carbonate (1.0 g) in dimethyl formamide (5 mL) was added a solution of 4H-benzo[1,4]oxazin-3-one (250 mg) in dimethyl formamide (3 mL) was added. After stirring in nitrogen atmosphere for 30 minutes another solution of Methyl-5-(4-iodo-butyl)-2-methyl-[1,3]dioxane-2-carboxylate (574 mg) in dimethyl formamide (3 mL) was added and the reaction mixture was stirred at ambient temperature for 2 hours. Reaction mixture was poured into ice cold water (50 mL) and extracted with ethyl acetate (3×30 mL). The combined organic extract was washed with water (50 mL), brine solution (50 mL), dried over sodium sulfate and evaporated under reduced pressure. Crude product was flash chromatographed over silica gel using 15% ethyl acetate in petroleum ether as eluent to yield 483 mg of product.

### Preparation 4

### Methyl 5-[4-(4-benzyloxy-phenyl)-butyl]-2-methyl-[1,3]dioxane-2-carboxylate (compound No. 18)

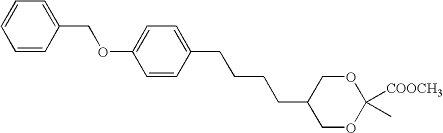

To a solution of 2-[4-(4-benzyloxy-phenyl)-butyl]-propane-1,3-diol (1.5 g) in acetonitrile (15 mL) was added methyl pyruvate (2.1 g) followed by 98% boron trifluoride-diethyl ether complex (1.7 g) and the reaction mixture was stirred at ambient temperature for 18 hours. The reaction mixture was poured into a saturated solution of sodium bicarbonate (100 mL) and extracted with ethyl acetate (3×50 mL). The combined organic extract was washed with water (100 mL), brine solution (100 mL), dried over sodium sulfate and evaporated under reduced pressure. Crude product was flash chromatographed over silica gel using 10% ethyl acetate in petroleum ether as eluent to obtain 867 mg of title product.

TABLE 1
A—(CH₂)ₘ—X—(CH₂)ₙ—B

| | | | | Mol. | % |
|---|---|---|---|---|---|
| S. No. | A | B | —(CH₂)ₘ—X—(CH₂)ₙ— | Wt. | Yield |
| 1. | 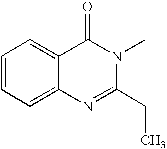 | 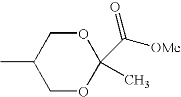 | (CH₂)₄ | 388 | 60 |
| | ¹H: 1.1 (2H, m), 1.2 (2H, m), 1.4 (3H, t, J=7.3 Hz), 1.5 (3H, s), 1.7 (2H, m), 2.0 (1H, m), 2.8 (2H, q, J=7.3 Hz), 3.4 (2H, t, | | | | |
| | J=11.5 Hz), 3.8 (3H, s), 4.0 (2H, dd, J=11.8 & 4.5 Hz), 4.06 (2H, t, J=7.7 Hz), 7.4 (1H, t, J=7.6 Hz), 7.6 (1H, d, J=7.7 Hz), 7.7 | | | | |
| | (1H, t, J=7.0 HZ), 8.2 (1H, d, J=7.8 Hz). | | | | |
| 2. | 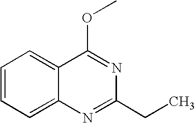 | 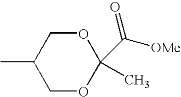 | (CH₂)₄ | 388 | 40 |
| | ¹H: 1.1 (2H, m), 1.2 (2H, m), 1.4 (3H, t, J=7.6 Hz), 1.5 (3H, s), 1.8 (2H, m), 2.0 (1H, m), 2.9 (2H, q, J=7.6 Hz), 3.4 (2H, t, | | | | |
| | J=11.5 Hz), 3.8 (3H, s), 4.0 (2H, dd, J=11.9 & 4.5 Hz), 4.5 (2H, t, J=6.5 Hz), 7.5 (1H, t, J=7.2 Hz), 7.8 (1H, t, J=7.1 Hz), 7.8 | | | | |
| | (1H, d, J=8.3 Hz), 8.1 (1H, d, J=8.0 Hz). | | | | |
| 3. | 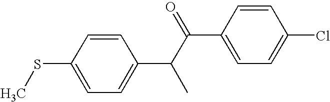 | 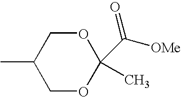 | (CH₂)₄ | 490.5 | 48 |
| | ¹H: 1.0 (2H, m), 1.2 (2H, m), 1.49 (3H, s), 1.7 (2H, m), 1.9 (1H, m), 2.0 (2H, m), 2.4 (3H, s), 3.3 (2H, t, J=11.6 Hz), 3.8 (2H, | | | | |
| | s), 3.9 (2H, dd, J=11.9 & 4.56 Hz), 4.3 (1H, t, J=7. 17 Hz), 7.1 (4H, m), 7.3 (2H, d, J=8.5 Hz), 7.8 (2H, d, J=8.5 Hz). | | | | |
| 4. | 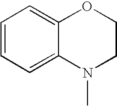 | 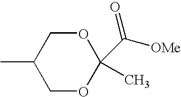 | (CH₂)₄ | 349 | 54 |
| | ¹H: 1.08 (2H, m), 1.3 (2H, m), 1.58 (3H, s), 1.75 (2H, m), 2.0 (2H, m), 3.2 (2H, t, J=7.5 Hz), 3.3 (2H, m), 3.4 (2H, t, J=11.6 Hz), | | | | |
| | 3.83 (3H, s), 3.96 (2H, dd, J=4.6 & 11.8 Hz), 4.22 (2H, t, J=4.3 Hz), 6.6 (2H, m), 6.8 (2H, m). | | | | |
| 5. | 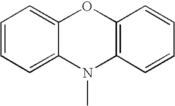 | 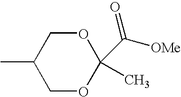 | (CH₂)₄ | 397 | 29 |
| | ¹H: 1.08 (2H, m), 1.36 (2H, m), 1.51 (3H, s), 1.65 (2H, m), 2.0 (2H, m), 3.4 (4H, m), 3.83 (3H, s), 3.9 (2H, dd, J=4.6 & 11.6 Hz), | | | | |
| | 6.4 (2H, d, J=7.86 Hz), 6.62 (4H, m), 6.77 (2H, m). | | | | |
| 6. | 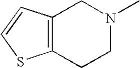 | 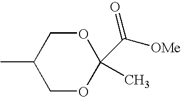 | (CH₂)₄ | 353 | 77 |
| | ¹H: 1.07 (2H, m), 1.3 (2H, m), 1.51 (3H, s),1.6 (2H, m), 2.03 (1H, m), 2.5 (2H, d, J=7.8 Hz), 2.76 (2H, t, J=5.0 Hz), 2.8 (2H, m), | | | | |
| | 3.39 (2H, t, J=11.64 Hz), 3.54 (2H, s), 3.53 (3H, s), 3.95 (2H, m), 6.7 (1H, d, J=5.0 Hz), 7.0 (1H, d, J=5.0 Hz). | | | | |
| 7. | 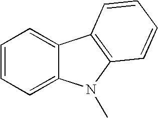 | 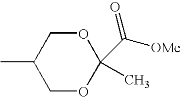 | (CH₂)₄ | 381 | 86 |
| | ¹H: 1.0 (2H, m), 1.3 (2H, m), 1.49 (3H, s), 1.87 (2H, m), 1.95 (1H, m), 3.4 (2H, t, J=11.63 Hz), 3.8 (3H, s), 3.89 (2H, dd, | | | | |
| | J=4.1 & 11.7 Hz), 4.3 (2H, t, J=7.0 Hz), 7.2 (2H, t, J=7.3 Hz), 7.34 (2H, d, J=8.1 Hz ,7.45 (2H, m), 8.1 (2H, d, J=7.74 Hz). | | | | |
| 8. | 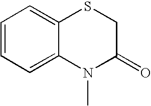 | 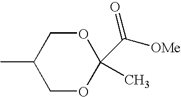 | (CH₂)₄ | 379 | 59 |
| | ¹H: 1.0 (2H, m), 1.28 (2H, m), 1.5 (3H, s), 1.54-1.68 (4H, m), 1.9 (1H, m), 3.36 (2H, s), 3.82 (3H, s), 3.89-4.01 (4H, m), | | | | |
| | 6.9 (2H, m), 7.2 (1H, d, J=7.2 Hz), 7.38 (1H, d, J=7.67 Hz). | | | | |
| 9. | 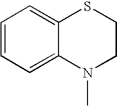 | 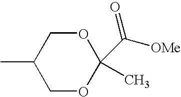 | (CH₂)₄ | 365 | 65 |
| | ¹H: 1.0 (2H, m), 1.3 (2H, m), 1.51 (3H, s), 1.58 (2H, m), 2.0 (1H, m), 3.0 (2H, m), 3.25 (2H, t, 17.5 Hz), 3.4 (2H, t, J=11.6 Hz), | | | | |
| | 3.6 (2H,t, J=5.0 Hz), 3.8 (3H, s), 3.94 (2H, dd, J=4.7 & 11.8 Hz), 6.6 (2H, m), 7.0 (2H, m). | | | | |
| 10. | 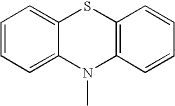 | 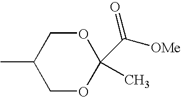 | (CH₂)₄ | 413 | 41 |
| | ¹H: 1.0 (2H, m), 1.35 (2H, m), 1.49 (3H, s), 1.75 (2H, m), 1.98 (1H, m), 3.33 (2H, t, J=11.6 Hz), 3.81 (3H, s), 3.9 (4H, m), 6.8 | | | | |
| | (2H, d, J=8.3 Hz), 6.9 (2H, t, J=7.5 Hz), 7.1 (4H, m). | | | | |
| 11. | 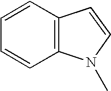 | 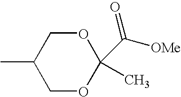 | (CH₂)₄ | 331 | 43 |
| | ¹H: 1.0 (2H, m), 1.2 (2H, m), 1.5 (3H, s), 1.7 (2H,m), 2.0 (1H, m), 3.3 (2H, t, J=11.6 Hz), 3.8 (3H, s), 3.9 (2H, dd, J=12.0 & | | | | |
| | 4.6 Hz), 4.1 (2H, t, J=6.9 Hz), 6.5 (1H, d, J=3.0 Hz), 7.0 (1H, d, J=3.1 Hz), 7.1 (1H, d, J=7.5 Hz), 7.2 (1H, t, J=7.2 Hz), 7.3 | | | | |
| | (1H, d, J=8.2 Hz), 7.6 (1H, d, J=7.8 Hz). | | | | |
| 12. | 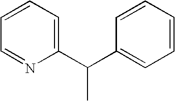 | 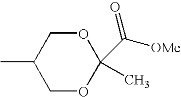 | (CH₂)₄ | 383 | 41 |
| | ¹H: 0.93-1.0 (2H, m), 1.24 (2H, m), 1.49 (3H, s), 1.52-1.62 (2H, m), 1.95-1.99 (2H, m), 2.06 (1H, m), 3.34 (2H,,t, J=11.55 Hz), | | | | |
| | 3.81 (3H, s), 3.90 (2H, q, J=11.9 & 4.5 Hz), 3.9 (1H, t, J=7.68 Hz), 7.05-7.39 (7H, m), 7.51-7.56 (1H, m), 8.55 (1H, t, | | | | |
| | J=5.04 Hz). | | | | |
| 13. | 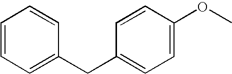 | 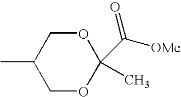 | (CH₂)₄ | 398 | 98 |
| | ¹H: 1.04-1.120 (2H, m), 1.37-1.47 (2H, m), 1.51 (3H, s), 1.68-1.80 (2H, m), 2.04 (1H, m), 3.40 (2H, t, J=11.65 Hz), 3.83 (3H, | | | | |
| | s), 3.89-3.99 (6H, m), 6.74-6.82 (2H, m), 7.04-7.10 (2H, m), 7.15-7.20 (3H, m), 7.27-7.30 (2H, m). | | | | |
| 14. | 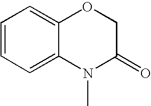 | 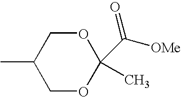 | (CH₂)₄ | 363 | 80 |
| | ¹H: 1.05-1.11 (2H, m), 1.25-1.38 (2H, m), 1.50 (3H, s), 1.58-1.71 (2H, m), 2.02 (1H, m), 3.39 (2H, t, J=11.67 Hz), 3.82 (3H, s), | | | | |
| | 3.88-3.97 (4H, m), 4.58 (2H, s), 6.92-7.04 (4H, m). | | | | |
| 15. | 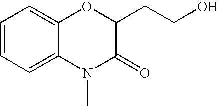 | 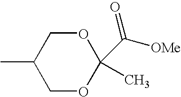 | (CH₂)₄ | 407 | 64 |
| | ¹H: 1.08 (2H, m), 1.34 (2H, m), 1.50 (3H, s), 1.63 (2H, m), 1.99 (1H, m), 2.19 (2H, m), 2.28 (1H, t, J=6.06 Hz exchangeable), | | | | |
| | 3.40 (2H, m), 3.82 (3H, s), 3.85-4.28 (6H, m), 4.67 (1H, t, J=6.52 Hz), 6.92-7.07 (4H, m). | | | | |
| 16. | 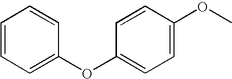 | 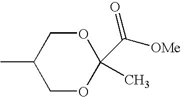 | (CH₂)₄ | 400 | 98 |
| 17. | 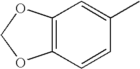 | 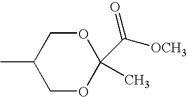 | (CH₂)₃ | 322 | 40 |
| | ¹H: 1.03 (2H, q, J=7.65 Hz), 1.5 (3H, s), 2.0-2.06 (1H, m), 2.50 (2H, t, J=7.5 Hz), 3.41 (2H, t, J=11.75 Hz), 3.49 (2H, t, J=5.13 | | | | |
| | Hz), 3.82 (3H, s), 3.82-3.96 (2H, dd, J=4.56 & 11.98 Hz), 5.92 (2H, s), 6.57 (1H, t, J=7.83 Hz), 6.62 (1H, s,), 6.72 (1H, | | | | |
| | d, J=7.83 Hz). | | | | |
| 18. | 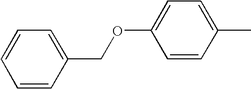 | 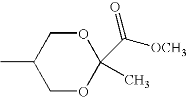 | (CH₂)₄ | 398 | 46 |
| | ¹H: 1.06 (2H, m), 1.22-1.32 (2H, m), 1.5 (3H, s), 1.59-1.61 (2H, m), 1.96-2.04 (1H, m), 2.52 (2H, t, J=7.6 Hz), 3.37 (2H, t, | | | | |
| | J=11.64 Hz), 3.82 (3H, s), 3.9-3.96 (2H, dd, J=4.53 & 11.93 Hz), 5.03 (2H, s), 6.88 (2H, d, J=8.13 Hz), 7.05 (2H, d, | | | | |
| | J=8.47 Hz), 7.29-7.44 (5H, m). | | | | |
| 19. | 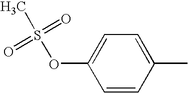 | 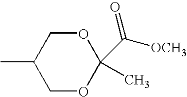 | (CH₂)₄ | 386 | 80 |
| 20. | 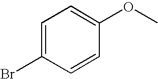 | 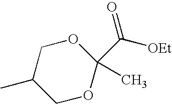 | (CH₂)₃ | 387 | 57 |
| | ¹H: 1.19-1.28 (2H, m), 1.34 (3H, t, J=7.11 Hz), 1.56 (3H, s), 1.71-1.76 (2H, m), 2.04-2.11 (1H, m), 3.41-3.49 (2H, m), | | | | |
| | 3.88 (2H, t, J=6.13 Hz), 3.96-3.99 (2H, m), 4.29 (2H, g, J=7.11 Hz), 6.72-6.78 (2H, m), 7.35 (2H, d, J=8.88 Hz). | | | | |
| 21. | 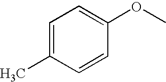 | 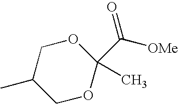 | (CH₂)₃ | 308 | 15 |
| 22. | 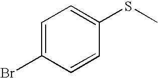 | 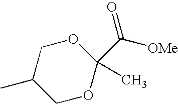 | (CH₂)₃ | 389 | 65 |
| | ¹H: 1.14-1.19 (2H, m), 1.5 (3H, s), 1.60-170 (2H, m), 2.01 (1H, m), 2.83≧2.94 (2H, m) , 3.39 (2H, m) 3.83 (3H, s), | | | | |
| | 3.92 (2H, dd, J=11.88 & 4.92 Hz), 7.15 (2H, d, J=8.37 Hz), 7.39 (2H, d, J=8.4 Hz). | | | | |
| 23. | 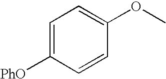 | 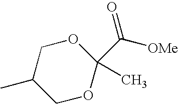 | (CH₂)₃ | 386 | 40 |
| 24. | 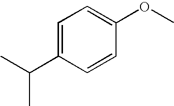 | 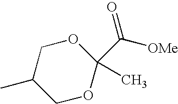 | (CH₂)₃ | 336 | 21 |
| 25. | 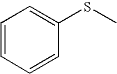 | 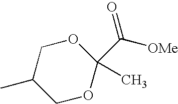 | (CH₂)₃ | 310 | 19 |
| 26. | 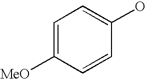 | 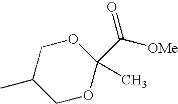 | 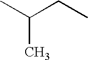 | 324 | 47 |
| 27. | 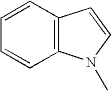 | 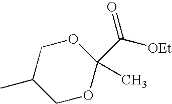 | (CH₂)₃ | 338 | 47 |
| 28. | 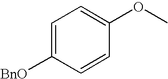 | 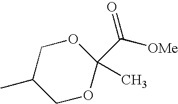 | (CH₂)₃ | 400 | 61 |
| 29. | 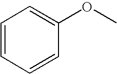 | 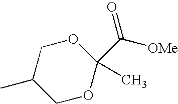 | (CH₂)₃ | 294 | 11 |
| 30. | 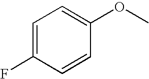 | 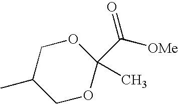 | (CH₂)₃ | 312 | 33 |
| 31. | 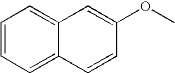 | 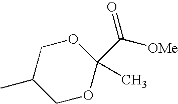 | (CH₂)₃ | 344 | 20 |
| 32. | 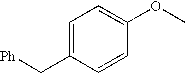 | 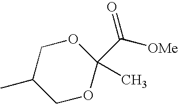 | (CH₂)₃ | 384 | 24 |

### Preparation 5

### 2-Methyl-5-(5-phenyl-5-pyridin-4-yl-pentyl)-[1,3]dioxane-2-carboxylic acid (compound No. 37)

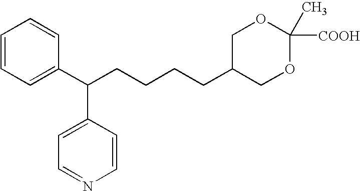

To a methanolic solution (10 mL) of methyl-[2-methyl-5-(5-phenyl-5-pyridin-4-yl-pentyl)-[1,3]dioxane-2-carboxylate (compound No. 12) (233 mg), prepared as in preparation 1 above was added a solution of sodium hydroxide (50 mg) in water (5 mL) and the reaction mixture was stirred at ambient temperature for 15 hours. The solvents were evaporated under reduced pressure and water (25 mL) was added to the residue. The mixture was acidified with 1 N hydrochloric acid and extracted with ethyl acetate (3×20 mL). The combined organic extract was washed with water (25 mL), brine (25 mL), dried over sodium sulfate and evaporated under reduced pressure. The thick gummy product obtained was triturated with petroleum ether to yield 150 mg of product.

### Preparation 6

### 5-[4-(4-Methanesulfonyloxy-phenyl)-butyl]-2-methyl-[1,3]dioxane-2-carboxylic acid (compound No. 50)

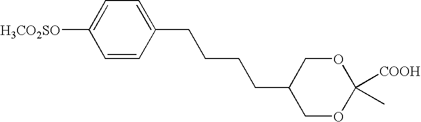

To a solution of Methyl-5-[4-(4-methanesulfonyloxy-phenyl)-butyl]-2-methyl-[1,3]dioxane-2-carboxylate (compound No. 19) (166 mg) in tetrahydrofuran (2 mL) was added another solution of lithium hydroxide (21 mg) in water (3 mL) and the reaction mixture was stirred at ambient temperature for 18 hours. Reaction mixture was diluted with water (20 mL), acidified to pH 2-3 with 1N hydrochloric acid and extracted with ethyl acetate (3×10 mL). The combined organic extract was washed with water (20 mL), brine solution (20 mL), dried over sodium sulfate and evaporated under reduced pressure to obtain 127 mg of product.

TABLE 2
A—(CH₂)ₘ—X—(CH₂)ₙ—B

| | | | | Mol. | % |
|---|---|---|---|---|---|
| S.No. | A | B | —(CH₂)ₘ—X—(CH₂)ₙ— | Wt. | Yield |
| 33. | 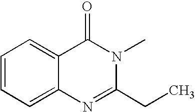 | 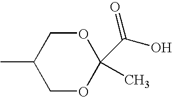 | (CH₂)₄ | 374 | 58 |
| | ¹H: 1.1 (2H, m), 1.2 (2H, m), 1.4 (3H, t, J=7.4 Hz), 1.52 (3H, s), 1.7 (2H, m), 2.0 (1H, m), 2.8 (2H, q, J=7.4 Hz), | | | | |
| | 3.5 (2H, t, J=11.5 Hz), 3.9 (2H, dd, J=11.8 & 4.5 Hz), 4.0 (2H, t, J=7.9 Hz), 7.4 (1H, t, J=7.3 Hz), 7.6 (1H, d, | | | | |
| | J=8.1 Hz), 7.7 (1H, t, J=7.2 Hz), 8.2 (1H, d, J=7.5 Hz). | | | | |
| 34. | 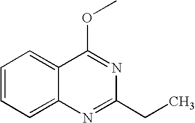 | 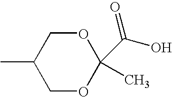 | (CH₂)₄ | 374 | 66 |
| | ¹H: 1.1 (2H, m), 1.2 (2H, m), 1.4 (3H, t, J=7.5 Hz), 1.5 (3H, s), 1.8 (2H, m), 2.0 (1H, s), 3.0 (2H, q, J=7.5 Hz), | | | | |
| | 3.5 (2H, t, J=11.5 Hz), 3.9 (2H, dd, J=11.7 & 4.3 Hz), 4.6 (2H, t, J=6.3 Hz), 7.5 (1H, t, J=7.3 Hz), 7.8 (1H, t, | | | | |
| | J=7.1. Hz), 7.9 (1H, d, J=8.4 Hz), 8.1 (1H, d, J=8.2 Hz). | | | | |
| 35. | 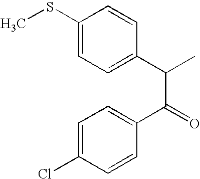 | 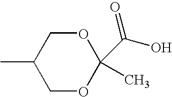 | (CH₂)₄ | 476.5 | 97 |
| | ¹H: 1.0 (2H, m), 1.2 (2H, m), 1.55 (3H, s), 1.7 (2H, m), 1.9-2.1 (3H, m), 2.4 (3H, s), 3.4 (2H, t, J=11.8 Hz), 3.9 | | | | |
| | (2H, dd, J=11.7 & 4.4 Hz), 4.3 (1H, t, J=7.15 Hz), 7.16 (4H, m), 7.3 (2H, d, J=8.46 Hz), 7.8 (2H, d, J=8.48 Hz). | | | | |
| 36. | 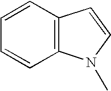 | 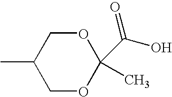 | (CH₂)₄ | 317 | 59 |
| | ¹H: 0.99-1.07 (2H, m), 1.21-1.32 (2H, m), 1.55 (3H, s), 1.76-1.85 (2H, m), 1.98 (1H, m), 3.41 (2H, t, J=11.59 | | | | |
| | Hz), 3.92 (2H, dd, J=12.0 & 4.5 Hz), 4.10 (2H, t, J=6.90 Hz), 6.48 (1H, d, J=3.03 Hz), 7.05 (1H, t, J=3.0 Hz), 7.10 | | | | |
| | (1H, d, J=7.12 Hz), 7.19 (1H, t, J=7.05 Hz), 7.30 (1H, d, J=8.19 Hz), 7.62 (1H, d, J=7.83 Hz). | | | | |
| 37. | 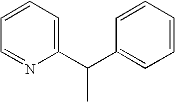 | 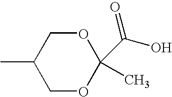 | (CH₂)₄ | 369 | 55 |
| | ¹H: 0.96 (2H, d, 16.72 Hz), 1.27 (2H, d, J=6.66 Hz), 1.59 (3H, s), 2.10 (5H, m), 3.43-3.52 (2H, m), 3.93 (2H, q, | | | | |
| | J=11.55 & 4.26 Hz), 4.28 (1H, t, J=7.65 Hz), 7.14-7.23 (3H, m), 7.31 (4H, m), 7.65 (1H, t, J=7.74 Hz), 8.63 (1H, | | | | |
| | d, J:=4.74 Hz) | | | | |
| 38. | 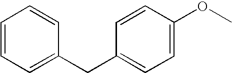 | 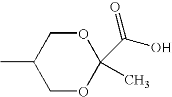 | (CH₂)₄ | 384 | 40 |
| | ¹H: 1.10-1.15 (2H, m), 1.38-1.48 (2H, m), 1.57 (3H, s), 1.63-1.78 (2H, m), 2.05 (1H, m), 3.46 (2H, t, J=11.15 | | | | |
| | Hz), 3.88-3.94 (4H, m), 3.97-4.02 (2H, dd, J=11.97 & 4.5 Hz), 6.77-6.80 (2H, d, J=8.49 Hz , 7.08 (2H, d, J=8.37 | | | | |
| | HZ), 7.15-7.29 (5H, m). | | | | |
| 39. | 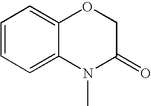 | 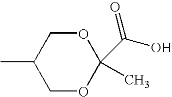 | (CH₂)₄ | 349 | 48 |
| | ¹H: 1.07-1.15 (2H, m), 1.29-1.34 (2H, m), 1.57 (3H, s), 1.59-1.69 (2H, m), 2.03 (1H, m), 3.47 (2H, t, J=11.49 | | | | |
| | Hz), 3.89-4.02 (4H, m), 4.59 (2H, s), 6.93-7.05 (4H, m). | | | | |
| 40. | 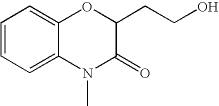 | 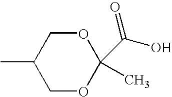 | (CH₂)₄ | 393 | 24 |
| | ¹H: 1.12 (2H, m), 1.28 (2H, m), 1.55 (3H, s), 1.64 (2H, dd, J=7.27 & 14.57 Hz), 2.03 (1H, m), 2.20 (2H, m), 3.48 | | | | |
| | (2H, m), 3.91 (6H, m), 4.67 (1H, t, J=6.44 Hz), 6.99 (4H, m). | | | | |
| 41. | 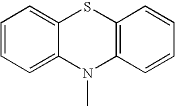 | 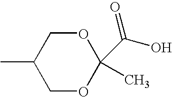 | (CH₂)₄ | 399 | 66 |
| | ¹H: 1.0 (2H, m), 1.36 (2H, m), 1.5 (3H, s), 1.76 (2H, m), 2.0 (1H, m), 3.4 (2H, t, J=11.5 Hz), 3.9 (2H, m), 3.96 | | | | |
| | (2H, dd, J=4.5 & 11.9 Hz), 6.84 (2H, m), 6.91 (2H, m), 7.15 (4H, d, J=7.0 Hz). | | | | |
| 42. | 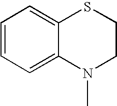 | 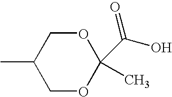 | (CH₂)₄ | 351 | 84 |
| | ¹H: 1.1 (2H, m), 1.3 (2H, m), 1.57 (3H, s), 1.6 (2H, m), 2.05 (1H, m), 3.0 (2H, dd, J=3.0 & 5.2 Hz), 3.25 (2H, t, | | | | |
| | J=7.5 Hz), 3.47 (2H, t, J=11.5 Hz), 3.6 (2H, dd, J=5.1 & 7.0 Hz), 4.0 (2H, dd, J=4.44 & 11.9 Hz), 6.6 (2H, m), | | | | |
| | 7.0 (2H, m). | | | | |
| 43. | 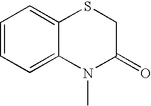 | 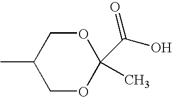 | (CH₂)₄ | 365 | 93 |
| | ¹H: 1.1 (2H, m), 1.3 (2H, m), 1.56 (3H, s), 1.6 (2H, m), 2.0 (1H, m), 2.4 (1H, bs), 3.37 (2H, s), 3.48 (2H, t, | | | | |
| | J=11.5 Hz), 4.0 (4H, m), 7.0 (2H, m), 7.2 (1H, m), 7.38 (1H, d, J=7.5 Hz). | | | | |
| 44. | 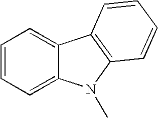 | 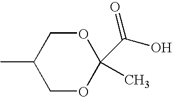 | (CH₂)₄ | 367 | 74 |
| | ¹H: 1.04 (3H, m), 1.3 (2H, m), 1.53 (3H, s), 1.84 (2H, m), 1.95 (1H, m), 3.4 (2H, t, J=11.5 Hz), 3.9 (2H, dd, | | | | |
| | J=4.4 & 11.8 Hz), 4.3 (2H, t, J=7.0 Hz), 7.2 (2H, t, J=7.3 Hz), 7.34 (2H, d, J=8.1 Hz), 7.45 (2H, m), 8.1 (2H, d, | | | | |
| | J=7.71 Hz). | | | | |
| 45. | 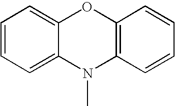 | 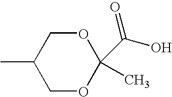 | (CH₂)₄ | 383 | 70 |
| | ¹H: 1.11 (2H, m), 1.4 (2H, m), 1.57 (3H, m), 1.6 (2H, m), 2.0 (1H, m), 3.5 (4H, t, J=11.5 Hz), 4.0 (2H, dd, J=4.4 & | | | | |
| | 11.8 Hz), 6.4 (2H, d, J=7.84 Hz), 6.6 (4H, m), 6.77 (2H, m). | | | | |
| 46. | 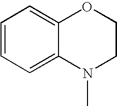 | 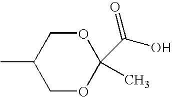 | (CH₂)₄ | 335 | 56 |
| | ¹H: 1.08 (2H, m), 1.3 (2H, m), 1.52-1.62 (5H, m), 2.04 (1H, m), 3.2 (2H, t, J=7.4 Hz), 3.3 (2H, t, J=4.3 Hz), 4.7 | | | | |
| | (2H, t, J=11.6 Hz), 4.0 (2H, dd, J=4.4 & 11.8 Hz), 4.22 (2H, t, J=4.3 Hz), 6.6 (2H, d, J=7.5 Hz), 6.8 (2H, m). | | | | |
| 47. | 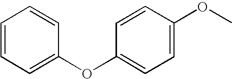 | 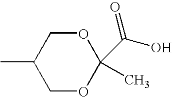 | 386 | 90 | |
| | ¹H: 1.1 (2H, m), 1.4 (2H, m), 1.5 (3H, s), 1.7 (2H, m), 2.0 (1H, m), 3.44-3.52 (2H, m), 3.9 (2H, t, J=6.09 Hz), 4.04 | | | | |
| | (2H, dd, J=4.41 & 11.85 Hz), 6.83 (2H, d, J=8.97 Hz), 6.92-6.97 (4H, m), 7.01-7.06 (1H, m), 7.30 (2H, d, | | | | |
| | J=7.95 Hz). | | | | |
| 48. | 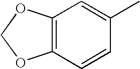 | 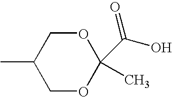 | (CH₂)₃ | 308 | 85 |
| | ¹H: 1.07 (2H, q, J=7.5 Hz), 1.48-1.54 (2H, m), 1.57 (3H, s), 2.01-2.08 (1H, m), 2.50 (2H, t, J=7.47 Hz), 3.45 | | | | |
| | (2H, t, J=11.58 Hz), 3.95-4.0 (2H, dd, J=4.46 & 11.95 Hz), 5.92 (2H, s), 6.59 (2H, t, J=8.92 Hz), 6.72 (1H, d, | | | | |
| | J=7.86 Hz). | | | | |
| 49. | 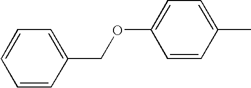 | 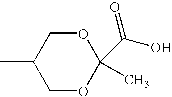 | (CH₂)₄ | 384 | 84 |
| | ¹H: 1.04-1.09 (2H, m), 1.23-1.33 (4H, m), 1.53 (3H, s), 1.97-2.05 (1H, m), 2.52 (2H, t, J=7.57 Hz), 3.43 (2H, t, | | | | |
| | J=11.52 Hz), 3.94-3.99 (2H, dd, J=4.49 & 11.8 Hz), 5.03 (2H, s), 6.8 (2H, d, J=8.4 Hz), 7.05 (2H, d, J=8.43 Hz), | | | | |
| | 7.29-7.44 (5H, m). | | | | |
| 50. | 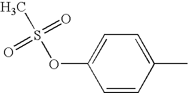 | 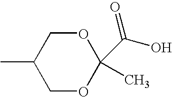 | (CH₂)₄ | 372 | 80 |
| | ¹H: 1.03-1.10 (2H, q, J=7.51 Hz), 1.28-1.34 (2H, m), 1.53 (3H, s), 1.6 (2H, m), 1.98-2.04 (1H, m), 2.59 (2H, t, | | | | |
| | J=7.56 Hz), 3.13 (3H, s), 3.44 (2H, t, J=11.65 Hz), 3.93-3.99 (2H, dd, J=4.68 & 11.94 Hz), 7.18 (4H, s). | | | | |
| 51. | 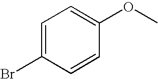 | 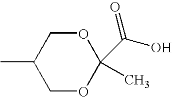 | (CH₂)₃ | 359 | 45 |
| | ¹H: 1.20-1.28 (2H, m), 1.58 (3H, s), 1.70-1.79 (2H, m), 2.05-2.13 (1H, m), 3.8 (2H, t, J=6.075 Hz), 4.0-4.05 | | | | |
| | (2H, m), 6.73 (2H, d, J=8.82 Hz), 7.35 (2H, d, J=8.85 Hz) | | | | |
| 52. | 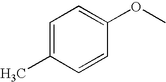 | 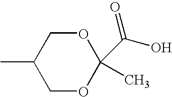 | (CH₂)₃ | 294 | 74 |
| | ¹H: 1.20-1.28 (2H, m), 1.58 (3H, s), 1.71-1.76 (2H, m), 2.1 (1H, m), 2.27 (3H, s), 3.46-3.54 (2H, m), 3.89 (2H, | | | | |
| | t, J=6.13 Hz), 4.03 (2H, dd, J=11.88 & 4.47 Hz), 6.76 (2H, d, J=8.43 Hz), 7.06 (2H, d, J=8.34 Hz). | | | | |
| 53. | 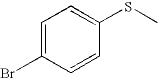 | 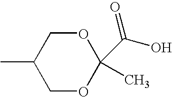 | (CH₂)₃ | 375 | 10 |
| | ¹H: 1.15-1.18 (2H, m), 1.5 (3H, s), 1.59-1.6 (2H, m), 1.99-2.06 (1H, m), 2.85 (2H, t, J=7.06 Hz), 3.42-3.49 (2H, | | | | |
| | m), 3.95 (2H, dd, J=11.88 & 4.34 Hz), 7.16 (2H, d, J=8.4 Hz), 7.39 (2H, d, J=8.43 Hz. | | | | |
| 54. | 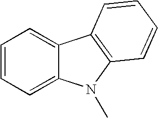 | 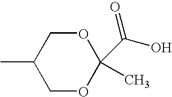 | (CH₂)₃ | 372 | 37 |
| | ¹H: 1.22-1.29 (2H, m), 1.72 (3H, s), 1.73-1.78 (2H, m), 2.04-2.14 (1H, m), 3.47-3.55 (2H, m), 3.9 (2H, t, | | | | |
| | J=6.09 Hz), 4.04 (2H, dd, J=4.41 & 11.85 Hz), 6.83 (2H, d, J=8.97 Hz), 6.92-6.97 (4H, m , 7.01-7.06 (1H, m), | | | | |
| | 7.30 (2H, d, J=7.95 Hz). | | | | |
| 55. | 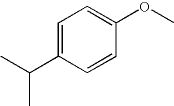 | 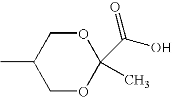 | (CH₂)₃ | 322 | 67 |
| | ¹H: 1.22 (6H, s), 1.25-1.28 (2H, m), 1.58 (3H, s), 1.69-1.78 (2H, m), 2.1 (1H, m), 2.80-2.89 (1H, m), 3.46-3.54 | | | | |
| | (2H, m), 3.90 (2H, t, J=6.1 Hz), 4.02 (2H, dd, J=4.62 & 11.82 Hz), 6.79 (2H, d, J=8.52 Hz), 7.12 (2H, d, | | | | |
| | J=8.52 Hz). | | | | |
| 56. | 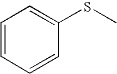 | 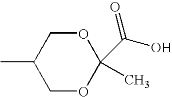 | (CH₂)₃ | 296 | 77 |
| | ¹H: 1.12-1.04 (2H, m), 1.31 (3H, s), 1.44-1.54 (2H, m), 1.83-1.84(1H, m), 2.90 (2H, t, J=7.1 Hz), 3.24-3.32 | | | | |
| | (2H, m), 3.79 (2H, dd, J=4.2 & 11.46 Hz), 7.13-7.29 (5H, m). | | | | |
| 57. | 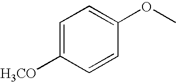 | 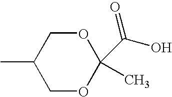 | (CH₂)₃ | 310 | 82 |
| | ¹H: 1.20-1.28 (2H, q, J=7.6 Hz), 1.58 (3H, s), 1.68-1.77 (2H, m), 2.07-2.13 (1H, m), 3.5 (2H, t, J=11.5 Hz), | | | | |
| | 3.76 (3H, s), 3.87 (2H, t, J=6.1 Hz), 4.0-4.06 (2H, dd, J=4.4 & 4.5 Hz), 6.7 (2H, d, J=9.5 Hz), 6.83 (2H, d, | | | | |
| | J=4.57 Hz). | | | | |
| 58. | 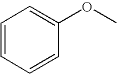 | 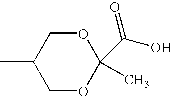 | (CH₂)₃ | 386 | 71 |
| | ¹H: 1.20-1.28 (2H, q, J=7.5 Hz), 1.58 (3H, s), 1.70-1.82 (2H, m), 2.05-2.11 (1H, m), 3.5 (1H, t, J=11.5 Hz), | | | | |
| | 3.82-4.0 (5H, m), 5.0 (2H, s), 6.77-6.91 (4H, m), 7.25 (1H, s), 7.28-7.42 (4H, m). | | | | |
| 59. | 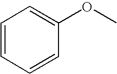 | 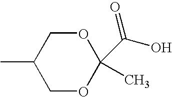 | (CH₂)₃ | 280 | 55 |
| | ¹H: 1.07-1.14 (2H, q, J=7.5 Hz), 1.32 (3H, s), 1.60-1.69 (2H, m), 1.84-1.92 (1H, m), 3.3-3.36 (2H, m), 3.84-3.95 | | | | |
| | (4H, m), 6.89 (3H, t, J=6.51 Hz), 7.25 (2H, t, J=7.89 Hz). | | | | |
| 60. | 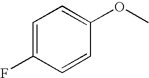 | 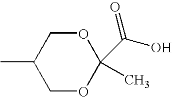 | (CH₂)₃ | 298 | 82 |
| | ¹H: 1.21-1.29 (2H, m), 1.59 (3H, s), 1.70-1.79 (2H, m), 2.0-2.14 (1H, m), 3.47-23.5 (2H, m), 3.86-3.92 | | | | |
| | (2H, m), 4.10-4.06 (2H, dd, J=4.5 & 11.7 Hz), 6.77-6.82 (2H, m), 6.97 (2H, d J=8.7 Hz). | | | | |
| 61. | 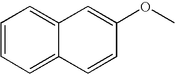 | 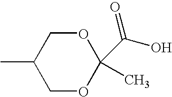 | (CH₂)₃ | 330 | 83 |
| | ¹H: 1.26-1.34 (2H, q, J=7.76 Hz), 1.59 (3H, s), 1.76-1.90 (2H, m), 2.10-2.17 (1H, m), 3.49-3.57 (2H, m), | | | | |
| | 4.0-4.12 (4H, m), 7.1 (2H, d, J=9 Hz), 7.32 (1H, t, J=7.23 Hz), 7.43 (1H, t, J=7.25 Hz), 7.69-7.7 (3H, m). | | | | |
| 62. | 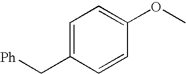 | 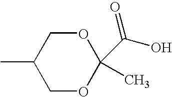 | (CH₂)₃ | 370 | 80 |

¹H: 1.20-1.27 (2H, q, J=7.6 Hz), 1.58 (3H, s), 1.58-1.71 (2H, m), 2.0-2.11 (1H, m), 3.46-3.54 (2H, m),
3.87-3.96 (4H, m), 3.99-4.0 (2H, dd, J=4.65 & 11.8 Hz), 6.8 (2H, d, J=7.72 Hz), 7.0 (2H, d, J=8.43 Hz), 7.17
(3H, d, J=7.73 Hz), 7.24-7.29 (2H, m).

### Preparation of Salts

Sodium and potassium salts of the compounds in table 2 were prepared by following the general procedure described below.

To a solution of carboxylic acid derivatives of the novel compounds (mentioned in table 2) (1 mmol) in alcoholic solvent like methanol, ethanol and the like was added another solution of sodium or potassium alkoxide (0.95 mmol) in alcoholic solvent and the reaction mixture was stirred for 3 hours at 25-30° C. The solvent was evaporated and the residue was triturated with dry diethyl ether or diisopropyl ether to obtain the salt of the corresponding carboxylic acid.

The compounds of the present invention lowered triglyceride, total cholesterol, LDL, VLDL and increased HDL and lowered serum glucose levels. This was demonstrated by in vivo animal experiments.

A) Demonstration of In vivo Efficacy of Compounds:

i) Serum triglyceride and total cholesterol lowering activity in Swiss albino mice:

Male Swiss albino mice (SAM) were bred in Zydus animal house. All these animals were maintained under 12 hour light and dark cycle at 25±1° C. Animals were given standard laboratory chow (NIN, Hyderabad, India) and water ad libitum. SAM of 20-30 g body weight range was used. The protocol approved by Institutional Animal Ethics Committee is being used.

The test compounds were administered orally to Swiss albino mice at 0.001 to 50 mg/kg/day dose for 6 days. The compound was administered after suspending it in 0.25% CMC or dissolving it in water, when compound is water-soluble. Control mice were treated with vehicle (0.25% of Carboxymethylcellulose; dose 10 ml/kg).

The blood samples were collected on 0^{th} day and in fed state 1 hour after drug administration on 6^{th} day of the treatment. The blood was collected in non heparinised capillary and the serum was analyzed for triglyceride and total cholesterol (Wieland, O. Methods of Enzymatic analysis. Bergermeyer, H., O., Ed., 1963. 211-214; Trinder, P. Ann. Clin. Biochem. 1969. 6: 24-27). Measurement of serum triglyceride and total cholesterol was done using commercial kits (Zydus-Cadila, Pathline, Ahmedabad, India).

Formula For Calculation:

Percentage reduction in triglycerides/total cholesterol were calculated according to the formula:

$$Percentage reduction ( \% ) = 1 - [ \frac{TT / OT}{TC / OC} ] \times 100$$

OC=Zero day control group value OT=Zero day treated group value TC=Test day control group TT=Test day treated group

TABLE 1
Triglyceride lowering activity in Swiss albino mice:

| Example No. | Dose (mg/kg/day) | % Triglyceride lowering |
|---|---|---|
| 50 | 10 | 32 |
| 38 | 10 | 32 |
| 49 | 10 | 26 |

ii) Surem triglyceride and total cholesterol lowering activity in Hamster of Syrian golden stain:

Male and Female Hamster of Syrian golden stain were bred in Zydus animal house. All these animals were maintained under 12-hour light and dark cycle at 22±3 degree C. The protocol approved by Institutional Animal Ethics Committee is being used. Two groups of animals were put on HF-HC (High fat and high cholesterol) diet for 14 days. On day 14 all the BF-HC diet whereas one group of animals of were put on normal diet for two weeks.

One group of animals on HF-HC diet were treated (po) with compounds of the present invention, at 0.001 to 50 mg/kg daily for 15 days while the other group received the vehicle. After 15 days blood samples. were be collected in non heparinized capillary from animals for determination of total cholesterol (TC), triglyceride (TG) (Wieland, O. Methods of Enzymatic analysis. Bergermeyer, H., O., Ed., 1963. 211-214; Trinder, P. Ann. Clin. Biochem. 1969. 6: 24-27). Measurement of serum triglyceride and total cholesterol was done using commercial kits (Pointe Scientific. Inc. USA.)

Formula For Calculation:

Percentage reduction in triglycerides/total cholesterol were calculated according to the formula:

Percentage reduction (%)=(TT−TC)/TC*100

TC=Test day control group TT=Test day treated group.

TABLE 2

| Example No. | Dose (mg/kg/day) | % Triglyceride lowering |
|---|---|---|
| 50 | 3 | 45 |

No adverse effects were observed for any of the mentioned compounds of invention. The compounds of the present invention showed good serum glucose, lipid and cholesterol lowering activity in the experimental animals used. These compounds are useful for the testing/prophylaxis of diseases caused by hyperlipidemia, hypercholesterolemia, hyperinsulinemia, hyperglycemia such as NIDDM, cardiovascular diseases, stroke, hypertension, obesity since such diseases are interlinked to each other.

1. (canceled)

2. A compound as claimed in claim 12 wherein, when ‘A’ is substituted, suitable substitutions on ‘A’ may be selected from hydroxyl, oxo, halo, thio, nitro, amino, cyano, formyl, or substituted or unsubstituted groups selected from amidino, alkyl, haloalkyl, perhaloalkyl, alkoxy, haloalkoxy, perhaloalkoxy, alkenyl, alkynyl, cycloalkyl, cycloalkenyl, bicycloalkyl, bicycloalkenyl, alkoxy, alkenoxy, cycloalkoxy, aryl, aryloxy, aralkyl, aralkoxy, acyl, acyloxy, acylamino, monosubstituted or disubstituted amino, arylamino, aralkylamino, carboxylic acid and its derivatives such as esters and amides, carbonylamino, hydroxyalkyl, aminoalkyl, alkoxyalkyl, aryloxyalkyl, aralkoxyalkyl, alkylthio, thioalkyl, arylthio, alkylsulfonylamino, alkylsulfonyloxy, alkoxycarbonylamino, aryloxycarbonylamino, aralkyloxycarbonylamino, aminocarbonylamino, alkylaminocarbonylamino, alkoxyamino, hydroxyl amino, sulfenyl derivatives, sulfonyl derivatives.

3. A compound as claimed in claim 12 wherein suitable substitutions on ‘B’ may be selected from hydroxyl, oxo, halo, thio, nitro, amino, cyano, formyl, or substituted or unsubstituted groups selected from alkyl, haloalkyl, aryl groups.

4. A compound as claimed in claim 12 wherein, the substitutions on any of the substituents on ‘A’ & ‘B’ may be selected from hydroxyl, oxo, halo, thio, nitro, amino, cyano, formyl, or substituted or unsubstituted groups selected from amidino, alkyl, haloalkyl, perhaloalkyl, alkoxy, haloalkoxy, perhaloalkoxy, alkenyl, alkynyl, cycloalkyl, cycloalkenyl, bicycloalkyl, bicycloalkenyl, alkoxy, alkenoxy, cycloalkoxy, aryl, aryloxy, aralkyl, aralkoxy, acyl, acyloxy, acylamino, monosubstituted or disubstituted amino, arylamino, aralkylamino, carboxylic acid and its derivatives such as esters and amides, carbonylamino, hydroxyalkyl, aminoalkyl, alkoxyalkyl, aryloxyalkyl, aralkoxyalkyl, alkylthio, thioalkyl, arylthio, alkylsulfonylamino, alkylsulfonyloxy, alkoxycarbonylamino, aryloxycarbonylamino, aralkyloxycarbonylamino, aminocarbonylamino, alkylaminocarbonylamino, alkoxyamino, hydroxyl amino, sulfenyl derivatives, sulfonyl derivatives.

5. The compounds of claim 12 selected from
Methyl-5-[4-(2-ethyl-4-oxo-4H-quinazolin-3-yl)-butyl]-2-methyl-[1,3]dioxane-2-carboxylate;
Methyl-5-[4-(2-ethyl-quinazolin-4-yloxy)-butyl]-2-methyl-[1,3]dioxane-2-carboxylate;
Methyl-5-[6-(4-chloro-phenyl)-5-(4-methylsulfanyl-phenyl)-6-oxo-hexyl]-2-methyl-[1,3]dioxane-2-carboxylate;
Methyl-5-[4-(2,3-dihydro-benzo[1,4]oxazin-4-yl)-butyl]-2-methyl-[1,3]dioxane-2-carboxylate;
Methyl-2-methyl-5-(4-phenoxazin-10-yl-butyl)-[1,3]dioxane-2-carboxylate;
Methyl-5-[4-(6,7-dihydro-4H-thieno[3,2-c]pyridin-5-yl)-butyl]-2-methyl-[1,3]dioxane-2-carboxylate;
Methyl-5-(4-carbazol-9-yl-butyl)-2-methyl-[1,3]dioxane-2-carboxylate;
Methyl-2-methyl-5-[4-(3-oxo-2,3-dihydro-benzo[1,4]thiazin-4-yl)-butyl]-[1,3]dioxane-2-carboxylate;
Methyl-5-[4-(2,3-dihydro-benzo[1,4]thiazin-4-yl)-butyl]-2-methyl-[1,3]dioxane-2-carboxylate;
Methyl-2-methyl-5-(4-phenothiazin-10-yl-butyl)-[1,3]dioxane-2-carboxylate;
Methyl-5-(4-indol-1-yl-butyl)-2-methyl-[1,3]dioxane-2-carboxylate;
Methyl-2-methyl-5-(5-phenyl-5-pyridin-4-yl-pentyl)-[1,3]dioxane-2-carboxylate;
Methyl-5-[4-(4-benzyl-phenoxy)-butyl]-2-methyl-[1,3]dioxane-2-carboxylate;
Methyl-2-methyl-5-[4-(3-oxo-2,3-dihydro-benzo[1,4]oxazin-4-yl)-butyl]-[1,3]dioxane-2-carboxylate;
Methyl-5-{4-[2-(2-hydroxy-ethyl)-3-oxo-2,3-dihydro-benzo[1,4]oxazin-4-yl]-butyl}-2-methyl-[1,3]dioxane-2-carboxylate;
Methyl-2-methyl-5-[4-(4-phenoxy-phenoxy)-butyl]-[1,3]dioxane-2-carboxylate;
Methyl-5-(3-benzo[1,3]dioxol-5-yl-propyl)-2-methyl-[1,3]dioxane-2-carboxylate;
Methyl-5-[4-(4-methanesulfonyloxy-phenyl)-butyl]-2-methyl-[1,3]dioxane-2-carboxylate;
Methyl-5-[4-(4-benzyloxy-phenyl)-butyl]-2-methyl-[1,3]dioxane-2-carboxylate;
Methyl-2-methyl -5-(3-phenylsulfanyl-propyl)-[1,3]dioxane-2-carboxylate;
Ethyl-5-[3-(4-bromo-phenoxy)-propyl]-2-methyl-[1,3]dioxane-2-carboxylate;
Methyl-2-methyl-5-[3-(4-phenoxy-phenoxy)-propyl]-[1,3]dioxane-2-carboxylate;
Methyl-5-[3-(4-isopropyl-phenoxy)-propyl]-2-methyl-[1,3]dioxane-2-carboxylate;
Methyl-2-methyl-5-(3-p-tolyloxy-propyl)-[1,3]dioxane-2-carboxylate;
Methyl-5-[3-(4-bromo-phenylsulfanyl)-propyl]-2-methyl-[1,3]dioxane-2-carboxylate;
Methyl-2-methyl-5-(3-phenoxy-propyl)-[1,3]dioxane-2-carboxylate;
Methyl-5-[3-(4-fluoro-phenoxy)-propyl]-2-methyl-[1,3]dioxane-2-carboxylate;
Methyl-2-methyl-5-[3-(naphthalen-2-yloxy)-propyl]-[1,3]dioxane-2-carboxylate;
Methyl-5-[3-(4-benzyloxy-phenoxy)-propyl]-2-methyl-[1,3]dioxane-2-carboxylate;
Methyl-5-[3-(4-methoxy-phenoxy)-propyl]-2-methyl-[1,3]dioxane-2-carboxylate;
Methyl-5-[3-(4-benzyl-phenoxy)-propyl]-2-methyl-[1,3]dioxane-2-carboxylate;
5-[4-(2-Ethyl-4-oxo-4H-quinazolin-3-yl)-butyl]-2-methyl-[1,3]dioxane-2-carboxylic acid and its pharmaceutically acceptable salts;
5-[4-(2-Ethyl-quinazolin-4-yloxy)-butyl]-2-methyl-[1,3]dioxane-2-carboxylic acid and its pharmaceutically acceptable salts;
5-[6-(4-Chloro-phenyl)-5-(4-methylsulfanyl-phenyl)-6-oxo-hexyl]-2-methyl-[1,3]dioxane-2-carboxylic acid and its pharmaceutically acceptable salts;
5-[4-(2,3-Dihydro-benzo[1,4]oxazin-4-yl)-butyl]-2-methyl-[1,3]dioxane-2-carboxylic acid and its pharmaceutically acceptable salts;
2-Methyl-5-(4-phenoxazin-10-yl-butyl)-[1,3]dioxane-2-carboxylic acid and its pharmaceutically acceptable salts;
5-(4-Carbazol-9-yl-butyl)-2-methyl-[1,3]dioxane-2-carboxylic acid and its pharmaceutically acceptable salts;
2-Methyl-5-[4-(3-oxo-2,3-dihydro-benzo[1,4]thiazin-4-yl)-butyl]-[1,3]dioxane-2-carboxylic acid and its pharmaceutically acceptable salts;
5-[4-(2,3-Dihydro-benzo[1,4]thiazin-4-yl)-butyl]-2-methyl-[1,3]dioxane-2-carboxylic acid and its pharmaceutically acceptable salts;
2-Methyl-5-(4-phenothiazin-10-yl-butyl)-[1,3]dioxane-2-carboxylic acid and its pharmaceutically acceptable salts;
5-(4-Indol-1-yl-butyl)-2-methyl-[1,3]dioxane-2-carboxylic acid and its pharmaceutically acceptable salts;
2-Methyl-5-(5-phenyl-5-pyridin-4-yl-pentyl)-[1,3]dioxane-2-carboxylic acid and its pharmaceutically acceptable salts;
5-[4-(4-Benzyl-phenoxy)-butyl]-2-methyl-[1,3]dioxane-2-carboxylic acid and its pharmaceutically acceptable salts;
2-Methyl-5-[4-(3-oxo-2,3-dihydro-benzo[1,4]oxazin-4-yl)-butyl]-[1,3]dioxane-2-carboxylic acid and its pharmaceutically acceptable salts;
5-{4-[2-(2-Hydroxy-ethyl)-3-oxo-2,3-dihydro-benzo[1,4]oxazin-4-yl]-butyl}-2-methyl-[1,3]dioxane-2-carboxylic acid and its pharmaceutically acceptable salts;
2-Methyl-5-[4-(4-phenoxy-phenoxy)-butyl]-[1,3]dioxane-2-carboxylic acid and its pharmaceutically acceptable salts;
5-(3-Benzo[1,3]dioxol-5-yl-propyl)-2-methyl-[1,3]dioxane-2-carboxylic acid and its pharmaceutically acceptable salts;
5-[4-(4-Methanesulfonyloxy-phenyl)-butyl]-2-methyl-[1,3]dioxane-2-carboxylic acid and its pharmaceutically acceptable salts;
5-[4-(4-Benzyloxy-phenyl)-butyl]-2-methyl-[1,3]dioxane-2-carboxylic acid and its pharmaceutically acceptable salts;
2-Methyl-5-(3-phenylsulfanyl-propyl)-[1,3]dioxane-2-carboxylic acid and its pharmaceutically acceptable salts;
5-[3-(4-Bromo-phenoxy)-propyl]-2-methyl-[1,3]dioxane-2-carboxylic acid and its pharmaceutically acceptable salts;
2-Methyl-5-[3-(4-phenoxy-phenoxy)-propyl]-[1,3]dioxane-2-carboxylic acid and its pharmaceutically acceptable salts;
5-[3-(4-isopropyl-phenoxy)-propyl]-2-methyl-[1,3]dioxane-2-carboxylic acid and its pharmaceutically acceptable salts;
2-Methyl-5-(3-p-tolyloxy-propyl)-[1,3]dioxane-2-carboxylic acid and its pharmaceutically acceptable salts;
5-[3-(4-Bromo-phenylsulfanyl)-propyl]-2-methyl-[1,3]dioxane-2-carboxylic acid and its pharmaceutically acceptable salts;
2-Methyl-5-(3-phenoxy-propyl)-[1,3]dioxane-2-carboxylic acid and its pharmaceutically acceptable salts;
5-[3-(4-Fluoro-phenoxy)-propyl]-2-methyl-[1,3]dioxane-2-carboxylic acid and its pharmaceutically acceptable salts;
2-Methyl-5-[3-(naphthalen-2-yloxy)-propyl]-[1,3]dioxane-2-carboxylic acid and its pharmaceutically acceptable salts;
5-[3-(4-Benzyloxy-phenoxy)-propyl]-2-methyl-[1,3]dioxane-2-carboxylic acid and its pharmaceutically acceptable salts;
5-[3-(4-Methoxy-phenoxy)-propyl]-2-methyl-[1,3]dioxane-2-carboxylic acid and its pharmaceutically acceptable salts;
5-[3-(4-Benzyl-phenoxy)-propyl]-2-methyl-[1,3]dioxane-2-carboxylic acid and its pharmaceutically acceptable salts.

6. A pharmaceutical composition which comprises compounds of formula (I), as claimed in claim 12 and a pharmaceutically acceptable carrier, diluent, excipients or solvate.

7. A method of preventing or treating diseases caused by hyperlipidaemia, hypercholesteremia, hyperglycemia, obesity, impaired glucose tolerance, leptin resistance, insulin resistance, diabetic complications, comprising administering an effective, non-toxic amount of compound of formula (I) or suitable pharmaceutical composition as defined in claim 12 to a patient in need thereof.

8. The method according to claim 12, wherein the disease is type 2 diabetes, impaired glucose tolerance, dyslipidaemia, hypertension, obesity, atherosclerosis, hyperlipidaemia, coronary artery disease, cardiovascular disorders and other diseases wherein insulin resistance is the underlying pathophysiologal mechanism.

9. A medicine for treating/reducing any of the disease conditions described in any preceding claims which comprises administering a compound of formula (I), as defined in claim 12 and a pharmaceutically acceptable carrier, diluent, excipients or solvate to a patient in need thereof.

10. Use of compounds of formula (I), their pharmaceutical compositions and medicines containing them as defined in claim 12 as a medicament suitable for the treatment of diseases mentioned in claim 12.

11. A process for preparing compound of formula (I) comprising the steps of
i) reacting a compound of formula (II) with a compound of formula (III), where ‘R’ represents suitable alkyl group and all other symbols are as defined in claim 12, to obtain compounds of formula (Ia) 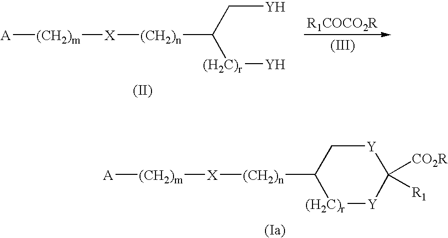
ii) alternatively, reacting a compound of formula (IV) with a compound of formula (V), where ‘L’ represents suitable leaving group and ‘R’ represents suitable alkyl group and all other symbols are as defined in claim 12, to obtain compound of formula (Ia), where all symbols are as defined in claim 12 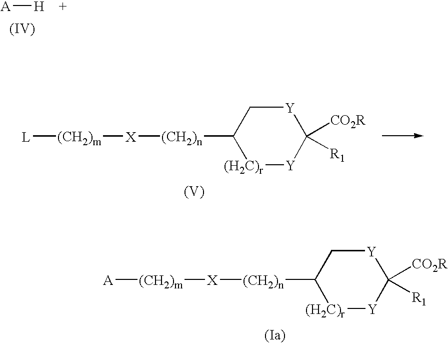
iii)converting the compound of formula (Ia) to compound of formula (I), where all symbols are as defined in claim 12

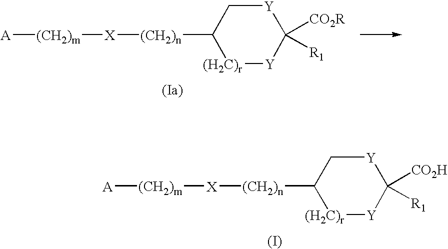

12. A compound of formula (I): 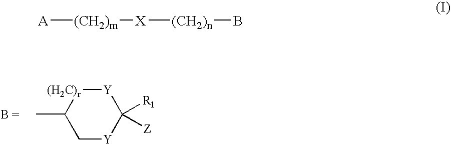
their tautomeric forms, their pharmaceutically acceptable salts, their pharmaceutically acceptable solvates, pharmaceutical compositions containing them, wherein ‘A’ represents optionally substituted, single or fused aryl, cycloalkyl group or an optionally substituted heteroaryl or an optionally substituted heterocyclyl group; ‘m’=0-2; ‘n’=3-6; ‘X’ represents O, S, —N-(Ra)- or —CH₂—; Ra represents H, linear or branched, group selected from alkyl, acyl or aryl, aralkyl group, which may optionally be substituted; ‘Y’ at each occurrence independently represent O or S; R₁ represents H, linear or branched substituted or unsubstituted alkyl; r=0-2; Z represents —(CH₂)ₛCOOH, alkoxycarbonyl, hydroxymethyl, —CN, substituted or unsubstituted tetrazoles, alkylcarbonyl groups, s=0-4; with the proviso that when ‘X’=CH₂ and
i) ‘A’ represents substituted aromatic heterocyclic group, the substitutions on ‘A’ does not represent aryl, aromatic, heterocyclic or cycloalkyl group; and
ii) ‘A’ represents substituted aryl group, the substituent on ‘A’ represents alkylsulfonyloxy, aryloxy, aralkoxy, cycloalkyl, heteroaryl or heterocyclic group.
